SEQUENCE LISTING (17 sequences)

SEQ ID NO: 1 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 2 - protein, 44 aa, Homo sapiens
MSDKPDMAEIEKFDKSKLKKTETQEKNPLPSKETIEQEKQAGES

SEQ ID NO: 3 - protein, 6 aa, Homo sapiens
LKKTET

SEQ ID NO: 4 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 5 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 6 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 7 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 8 - protein, 236 aa, Homo sapiens
MVSPDPQIIVVAPETELASMQVQRTEDGVTIIQIFWVGRKGELLRRTPVSSAMQTPMGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 9 - protein, 52 aa, Homo sapiens
MGIPMGKSMLVLLTFLAFASCCIAAYRPSETLCGGELVDTLQFVCGDRGFYF

SEQ ID NO: 10 - protein, 64 aa, Homo sapiens
QTPNEECLFLERLEENHYNTYISKKHAEKNWFVGLKKNGSCKRGPRTHYGQKAILFLPLPVSSD

SEQ ID NO: 11 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 12 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 13 - protein, 789 aa, Homo sapiens
MAATTANPEMTSDVPSLGPAIASGNSGPGIQGGGAIVQRAIKRRPGLDFDDDGEGNSKFLRCDDDQMSNDKERFARSDDEQSSADKERLARENHSEIERRRRNKMTAYITELSDMVPTCSALARKPDKLTILRMAVSHMKSLRGTGNTSTDGSYKPSFLTDQELKHLILEAADGFLFIVSCETGRVVYVSDSVTPVLNQPQSEWFGSTLYDQVHPDDVDKLREQLSTSENALTGRILDLKTGTVKKEGQQSSMRMCMGSRRSFICRMRCGSSSVDPVSVNRLSFVRNRCRNGLGSVKDGEPHFVVVHCTGYIKAWPPAGVSLPDDDPEAGQGSKFCLVAIGRLQVTSSPNCTDMSNVCQPTEFISRHNIEGIFTFVDHRCVATVGYQPQELLGKNIVEFCHPEDQQLLRDSFQQVVKLKGQVLSVMFRFRSKNQEWLWMRTSSFTFQNPYSDEIEYIICTNTNVKNSSQEPRPTLSNTIQRPQLGPTANLPLEMGSGQLAPRQQQQQTELDMVPGRDGLASYNHSQVVQPVTTTGPEHSKPLEKSDGLFAQDRDPRFSEIYHNINADQSKGISSSTVPATQQLFSQGNTFPPTPRPAENFRNSGLAPPVTIVQPSASAGQMLAQISRHSNPTQGATPTWTPTTRSGFSAQQVATQATAKTRTSQFGVGSFQTPSSFSSMSLPGAPTASPGAAAYPSLTNRGSNFAPETGQTAGQFQTRTAEGVGVWPQWQGQQPHHRSSSSEQHVQQPPAQQPGQPEVFQEMLSMLGDQSNSYNNEEFPDLTMFPPFSE

SEQ ID NO: 14 - protein, 37 aa, Homo sapiens [sequence omitted]

SEQ ID NO: 15 - protein, 390 aa, Homo Sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 16 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 17 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

(19) United States
(12) Patent Application Publication
(10) Pub. No.: US 2011/0280834 A1
(43) Pub. Date: Nov. 17, 2011

(54) METHODS AND COMPOSITIONS FOR CARDIAC TISSUE REGENERATION

(75) Inventors: James S. Forrester, Malibu, CA (US); Raj R. Makkar, Los Angeles, CA (US); Eduardo Marbán, Beverly Hills, CA (US)

(73) Assignee: CEDARS-SINAI MEDICAL CENTER, Los Angeles, CA (US)

(21) Appl. No.: 13/144,881

(22) PCT Filed: Jan. 15, 2010
(86) PCT No.: PCT/US10/21277

§ 371 Date: Jul. 15, 2011

Related U.S. Application Data
(60) Provisional application No. 61/145,490, filed on Jan. 16, 2009.

(51) Int. Cl. A61K 35/12; A61P 9/00; A61K 38/18; A61K 38/00

(52) U.S. Cl. 424/93.1; 514/16.4; 514/8.9

(57) ABSTRACT

Methods for treating an injured cardiac tissue in a subject are provided herein. Methods for improving survival, engraftment and proliferation of stem cells in a cardiac tissue are provided. Also provided are methods for generating cardiac cells. Further provided are compositions for generating cardiac cells in a subject.

### CROSS-REFERENCE TO RELATED APPLICATION

This application claims the U.S. Provisional Application No. 61/145,490, filed Jan. 16, 2009, the entire content of which is incorporated by reference herein.

### FIELD OF INVENTION

Provided herein, for example, are methods for improving survival of stem cells in a cardiac tissue. Also provided, for example, are methods for engraftment of stem cells in a cardiac tissue. Further provided are methods for improving proliferation of stem cells in a cardiac tissue. Also provided are methods for generating cardiac cells. Further provided are methods for treating an injured cardiac tissue in a subject. Also provided are compositions for generating cardiac cells in a subject.

### BACKGROUND

Heart disease is a leading cause of fatalities in modern societies. A major challenge for the treatment and prevention of heart disease is the limited capacity of cell regeneration in the cardiac tissue. To date, spontaneous cardiac cell regeneration in mammals has been reported only in the mutant Murphy Roth Large (MRL) mice (Leferovich et al. (2001) Proc. Natl. Acad. Sci. USA 98:9830). Although MRL mouse myocardium appears to have the capacity to regenerate, recent studies have shown that following extensive cryoablation and myocardial infarction induced by left coronary artery ligation, infarct size in the MRL mice is no different from that in the wild-type mice (Vela et al. (2008) Cardiovasc. Pathol. 17:1).

Stem cell therapy offers enormous potential for heart regeneration. However, stem cells that are transplanted into the cardiac tissue generally demonstrate low survival rate. For example, the transplanted cells generally show poor cell engraftment, inefficient proliferation and undergo inflammation and apoptosis quickly after being administered into the cardiac tissue. Thus, to date, the use of stem cells in cardiac repair has been limited.

Accordingly, there is a need in the art to provide methods and compositions that increase the vitality of stem cells used for cardiac repair.

### SUMMARY

Provided herein, for example, are methods for improving survival of stem cells in a cardiac tissue. Also provided, for example, are methods for engraftment of stem cells in a cardiac tissue. Further provided are methods for improving proliferation of stem cells in a cardiac tissue. Also provided are methods for generating cardiac cells. Further provided are methods for treating an injured cardiac tissue in a subject. Also provided are compositions for generating cardiac cells in a subject.

In several embodiments, there is provided a method for improving survival of stem cells in a cardiac tissue, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the cardiac tissue with the stem cells, such that survival of the stem cells is improved relative to survival of stem cells that have undergone (b) but not (a).

In one embodiment, provided herein is a method for improving survival of stem cells in a cardiac tissue, comprising: (a) contacting stem cells with a positive effector and a negative effector, wherein the positive effector is different from the negative effector; and (b) contacting the cardiac tissue with the stem cells, such that survival of the stem cells is improved relative to survival of stem cells that have undergone (b) but not (a).

In another embodiment, provided herein is a method for improving survival of stem cells in a cardiac tissue, comprising: (a) contacting stem cells with a positive effector and an ancillary effector, wherein the positive effector is different from the ancillary effector; and (b) contacting the cardiac tissue with the stem cells, such that survival of the stem cells is improved relative to survival of stem cells that have undergone (b) but not (a).

In yet another embodiment, provided herein is a method for improving survival of stem cells in a cardiac tissue, comprising: (a) contacting stem cells with a negative effector and an ancillary effector, wherein the negative effector is different from the ancillary effector; and (b) contacting the cardiac tissue with the stem cells, such that survival of the stem cells is improved relative to survival of stem cells that have undergone (b) but not (a).

In yet another embodiment, provided herein is a method for improving survival of stem cells in a cardiac tissue, comprising: (a) contacting stem cells with a positive effector, a negative effector and an ancillary effector, wherein the positive effector, the negative effector and the ancillary effector are different from one another; and (b) contacting the cardiac tissue with the stem cells, such that survival of the stem cells is improved relative to survival of stem cells that have undergone (b) but not (a).

In a several embodiments, there is provided a method for engraftment of stem cells in a cardiac tissue, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the cardiac tissue with the stem cells such that engraftment of the stem cells occurs.

In one embodiment, provided herein is a method for engraftment of stem cells in a cardiac tissue, comprising: (a) contacting stem cells with a positive effector and a negative effector, wherein the positive effector is different from the negative effector; and (b) contacting the cardiac tissue with the stem cells, such that engraftment of the stem cells occurs.

In another embodiment, provided herein is a method for engraftment of stem cells in a cardiac tissue, comprising: (a) contacting stem cells with a positive effector and an ancillary effector, wherein the positive effector is different from the ancillary effector; and (b) contacting the cardiac tissue with the stem cells, such that engraftment of the stem cells occurs.

In yet another embodiment, provided herein is a method for engraftment of stem cells in a cardiac tissue, comprising: (a) contacting stem cells with a negative effector and an ancillary effector, wherein the negative effector is different from the ancillary effector; and (b) contacting the cardiac tissue with the stem cells, such that engraftment of the stem cells occurs.

In yet another embodiment, provided herein is a method for engraftment of stem cells in a cardiac tissue, comprising: (a) contacting stem cells with a positive effector, a negative effector and an ancillary effector, wherein the positive effector, the negative effector and the ancillary effector are different from one another; and (b) contacting the cardiac tissue with the stem cells such that engraftment of the stem cells occurs.

In several embodiments, there is provided a method for improving proliferation of stem cells in a cardiac tissue, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the cardiac tissue with the stem cells, such that proliferation of the stem cells is improved relative to proliferation of stem cells that have undergone (b) but not (a).

In one embodiment, provided herein is a method for improving proliferation of stem cells in a cardiac tissue, comprising: (a) contacting stem cells with a positive effector and a negative effector, wherein the positive effector is different from the negative effector; and (b) contacting the cardiac tissue with the stem cells, such that proliferation of the stem cells is improved relative to proliferation of stem cells that have undergone (b) but not (a).

In another embodiment, provided herein is a method for improving proliferation of stem cells in a cardiac tissue, comprising: (a) contacting stem cells with a positive effector and an ancillary effector, wherein the positive effector is different from the ancillary effector; and (b) contacting the cardiac tissue with the stem cells, such that proliferation of the stem cells is improved relative to proliferation of stem cells that have undergone (b) but not (a).

In yet another embodiment, provided herein is a method for improving proliferation of stem cells in a cardiac tissue, comprising: (a) contacting stem cells with a negative effector and an ancillary effector, wherein the negative effector is different from the ancillary effector; and (b) contacting the cardiac tissue with the stem cells, such that proliferation of the stem cells is improved relative to proliferation of stem cells that have undergone (b) but not (a).

In yet another embodiment, provided herein is a method for improving proliferation of stem cells in a cardiac tissue, comprising: (a) contacting stem cells with a positive effector, a negative effector and an ancillary effector, wherein the positive effector, the negative effector and the ancillary effector are different from one another; and (b) contacting the cardiac tissue with the stem cells, such that proliferation of the stem cells is improved relative to proliferation of stem cells that have undergone (b) but not (a).

In several embodiments, provided herein is a method for generating cardiac cells in a subject, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting a cardiac tissue of the subject with the stem cells, such that cardiac cells are generated.

In one embodiment, provided herein is a method for generating cardiac cells in a subject, comprising: (a) contacting stem cells with a positive effector and a negative effector, wherein the positive effector is different from the negative effector; and (b) contacting a cardiac tissue of the subject with the stem cells, such that cardiac cells are generated.

In another embodiment, provided herein is a method for generating cardiac cells in a subject, comprising: (a) contacting stem cells with a positive effector and an ancillary effector, wherein the positive effector is different from the ancillary effector; and (b) contacting a cardiac tissue of the subject with the stem cells, such that cardiac cells are generated.

In yet another embodiment, provided herein is a method for generating cardiac cells in a subject, comprising: (a) contacting stem cells with a negative effector and an ancillary effector, wherein the negative effector is different from the ancillary effector; and (b) contacting a cardiac tissue of the subject with the stem cells, such that cardiac cells are generated.

In yet another embodiment, provided herein is a method for generating cardiac cells in a subject, comprising: (a) contacting stem cells with a positive effector, a negative effector and an ancillary effector, wherein the positive effector, the negative effector and the ancillary effector are different from one another; and (b) contacting a cardiac tissue of the subject with the stem cells, such that cardiac cells are generated.

In several embodiments, there is provided a method for treating an injured cardiac tissue in a subject, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the injured cardiac tissue with the stem cells, such that the cardiac tissue is treated.

In one embodiment, provided herein is a method for treating an injured cardiac tissue in a subject, comprising: (a) contacting stem cells with a positive effector and a negative effector, wherein the positive effector is different from the negative effector; and (b) contacting the injured cardiac tissue with the stem cells, such that the cardiac tissue is treated.

In another embodiment, provided herein is a method for treating an injured cardiac tissue in a subject, comprising: (a) contacting stem cells with a positive effector and an ancillary effector, wherein the positive effector is different from the ancillary effector; and (b) contacting the injured cardiac tissue with the stem cells, such that the cardiac tissue is treated.

In yet another embodiment, provided herein is a method for treating an injured cardiac tissue in a subject, comprising: (a) contacting stem cells with a negative effector and an ancillary effector, wherein the negative effector is different from the ancillary effector; and (b) contacting the injured cardiac tissue with the stem cells, such that the cardiac tissue is treated.

In yet another embodiment, provided herein is a method for treating an injured cardiac tissue in a subject, comprising: (a) contacting stem cells with a positive effector, a negative effector and an ancillary effector, wherein the positive effector, the negative effector and the ancillary effector are different from one another; and (b) contacting the injured cardiac tissue with the stem cells, such that the cardiac tissue is treated.

In several embodiments, there is provided a method for treating an injured cardiac tissue in a subject, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another, such that the cardiac tissue is treated.

In one embodiment, provided herein is a method for treating an injured cardiac tissue in a subject, comprising: contacting stem cells with a positive effector and a negative effector, wherein the positive effector is different from the negative effector, such that the cardiac tissue is treated.

In another embodiment, provided herein is a method for treating an injured cardiac tissue in a subject, comprising: (a) contacting stem cells with a positive effector and an ancillary effector, wherein the positive effector is different from the ancillary effector, such that the cardiac tissue is treated.

In yet another embodiment, provided herein is a method for treating an injured cardiac tissue in a subject, comprising: (a) contacting stem cells with a negative effector and an ancillary effector, wherein the negative effector is different from the ancillary effector, such that the cardiac tissue is treated.

In yet another embodiment, provided herein is a method for treating an injured cardiac tissue in a subject, comprising: (a) contacting stem cells with a positive effector, a negative effector and an ancillary effector, wherein the positive effector, the negative effector and the ancillary effector are different from one another, such that the cardiac tissue is treated.

In several embodiments, there is provided a method for treating an injured cardiac tissue in a subject, comprising: contacting the injured cardiac tissue with (a) cardiosphere-derived cells (CDCs); and (b)(i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another, such that the cardiac tissue is treated.

In one embodiment, provided herein is a method for treating an injured cardiac tissue in a subject, comprising: contacting the injured cardiac tissue with CDCs and a positive effector and a negative effector, wherein the positive effector is different from the negative effector such that the cardiac tissue is treated.

In another embodiment, provided herein is a method for treating an injured cardiac tissue in a subject, comprising: contacting the injured cardiac tissue with CDCs and a positive effector and an ancillary effector, wherein the positive effector is different from the ancillary effector such that the cardiac tissue is treated.

In yet another embodiment, provided herein is a method for treating an injured cardiac tissue in a subject, comprising: contacting the injured cardiac tissue with CDCs and a negative effector and an ancillary effector, wherein the negative effector is different from the ancillary effector, such that the cardiac tissue is treated.

In yet another embodiment, provided herein is a method for treating an injured cardiac tissue in a subject, comprising: contacting the injured cardiac tissue with CDCs and a positive effector, a negative effector and an ancillary effector, wherein the positive effector is different from the ancillary effector such that the cardiac tissue is treated.

In several embodiments, there is provided a method for treating an injured cardiac tissue in a subject, comprising: contacting the injured cardiac tissue with CDCs, adenosine, and at least one of thymosin β4 or periostin, such that the cardiac tissue is treated.

In several embodiments, there is provided a composition for generating cardiac cells in a subject, comprising: (a) CDCs; and (b)(i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another.

In one embodiment, provided herein is a composition for generating cardiac cells in a subject, comprising CDCs or induced pluripotent stem cells and a positive effector and a negative effector, wherein the positive effector is different from the negative effector.

In another embodiment, provided herein is a composition for generating cardiac cells in a subject, comprising CDCs or induced pluripotent stem cells and a positive effector and an ancillary effector, wherein the positive effector is different from the ancillary effector.

In yet another embodiment, provided herein is a composition for generating cardiac cells in a subject, comprising CDCs or induced pluripotent stem cells and a negative effector and an ancillary effector, wherein the negative effector is different from the ancillary effector.

In yet another embodiment, provided herein is a composition for generating cardiac cells in a subject, comprising CDCs or induced pluripotent stem cells and a positive effector, a negative effector and an ancillary effector, wherein the positive effector, the negative effector and the ancillary effector are different from one another.

According to several embodiments disclosed herein, a method for treating injured cardiac tissue in a subject is provided. In several embodiments, the method comprises identifying a subject having injured cardiac tissue, providing two or more of a positive effector, a negative effector, and an ancillary effector, providing non-embryonic cardiac stem cells, contacting the injured cardiac tissue or the stem cells with two or more of the effectors, and contacting the stem cells with the injured cardiac tissue, wherein the injured cardiac tissue has a deficiency in one or more of cardiac output, cardiac tissue viability, and/or cardiac blood flow, wherein contacting the stem cells with the injured cardiac tissue improves one or more of the cardiac tissue deficiencies, thereby treating the injured cardiac tissue, and wherein the positive effector, negative effector, and ancillary effector are different from one another. In one embodiment, the positive effector is characterized by the ability to activate, enhance, or promote one or more of cell proliferation, cell engraftment, cell migration, cell differentiation, or cell cycle re-entry. In one embodiment, the negative effector is characterized by the ability to inhibit or reduce one or more of apoptotic cell death or inflammation. In one embodiment, the ancillary effector promotes one or more of angiogenesis, revascularization, cell-to-cell contact, or cell-to-cell communication.

In some embodiments, the positive effector is selected from the group consisting of one or more of the following: periostin, thymosin beta-4, hepatocyte growth factor, insulin-like growth factor, fibroblast growth factor, and a transcription factor. In some embodiments, the negative effector is selected from the group consisting of one or more of the following: adenosine, an adenosine agonist, an adenosine receptor agonist, a phosphoinositide 3-kinase inhibitor, a caspase inhibitor, cyclosporine, an opiod receptor antagonist, pinacidil, a nitric oxide donor, poly(ADP-ribose) inhibitors, sodium-hydrogen exchange inhibitors, and thymosin beta-4. In some embodiments, the ancillary effector is further characterized by the ability to facilitate the effects of positive and/or negative effectors. In certain embodiments, the ancillary effector is selected from the group consisting of one or more of the following: p38 MAP kinase inhibitors, phosphodiesterase inhibitors, stem cell factor, and transforming growth factor beta.

In certain embodiments, the injured cardiac tissue or the stem cells are concurrently contacted with two or more of the effectors prior to being contacted with one another. In other embodiments, the injured cardiac tissue is sequentially contacted with the stem cells followed by two or more of the effectors. In yet other embodiments, the injured cardiac tissue is sequentially contacted with two or more of the effectors followed by the stem cells.

In several embodiments, the cardiac stem cells are cardiosphere derived cells (CDCs). In certain embodiments, the source of stem cells is autologous relative to the subject having injured cardiac tissue while in other embodiments, the source of stem cells is allogeneic relative to the subject having injured cardiac tissue. In certain embodiments, the stem cells are contacted with the injured cardiac tissue at a dose ranging from about 1×105 to 1×109 stem cells.

In several embodiments the stem cells and optionally one or more of the effectors are embedded into a biocompatible medium prior to contacting the stem cells with the injured cardiac tissue.

In certain embodiments, the subject having injured cardiac tissue is a human.

In several embodiments, treatment of the injured cardiac tissue results in an improvement in one or more of the cardiac tissue deficiencies as measured by one or more of preservation of injured cardiac tissue, regeneration of new cardiac tissue, increases in blood flow to the injured tissue, increases in myocardial perfusion, improvements in stroke volume, ejection fraction, cardiac output, ventricular wall thickening, segmental shortening and heart pumping.

In several embodiments disclosed herein, there is provided a method for treating injured cardiac tissue in a subject, comprising identifying a subject having injured cardiac tissue, providing two or more of a positive effector, a negative effector, and an ancillary effector, providing stem cells, contacting the injured cardiac tissue or the stem cells with two or more of the effectors, and contacting the stem cells with the injured cardiac tissue, wherein the injured cardiac tissue has a deficiency in one or more of cardiac output, cardiac tissue viability, cardiac blood flow, wherein the positive effector, negative effector, and ancillary effector are different from one another, and wherein the contacting of the stem cells with the injured cardiac tissue improves one or more of the cardiac tissue deficiencies, thereby treating the injured cardiac tissue.

In some embodiments, the stem cells are induced pluripotent stem cells, embryonic stem cells, cardiac stem cells, bone marrow stem cells, placenta-derived stem cells, amniotic stem cells, embryonic germ cells, or spermatocytes. In several embodiments, the stem cells are non-embryonic cells such as non-embryonic cardiac cells.

In some embodiments, the positive effector is characterized by the ability to activate, enhance, or promote one or more of cell proliferation, cell engraftment, cell migration, cell differentiation, or cell cycle re-entry, the negative effector is characterized by the ability to inhibit or reduce one or more of apoptotic cell death or inflammation, and the ancillary effector promotes one or more of angiogenesis, revascularization, cell-to-cell contact, or cell-to-cell communication.

In several embodiments disclosed herein, a composition for treating injured cardiac tissue in a subject is provided. The use of said compositions in the preparation a medicament for treating cardiac tissue is provided in several embodiments. In several embodiments, the composition comprises non-embryonic cardiac stem cells and two or more of a positive effector, a negative effector, and an ancillary effector. In one embodiment, the stem cells are cardiosphere-derived cells. In one embodiment, the positive effector, negative effector, and ancillary effector are different from one another. In one embodiment, the positive effector is characterized by the ability to activate, enhance, or promote one or more of cell proliferation, cell engraftment, cell migration, cell differentiation, or cell cycle re-entry. In one embodiment, the negative effector is characterized by the ability to inhibit or reduce one or more of apoptotic cell death or inflammation. In one embodiment, the ancillary effector promotes one or more of angiogenesis, revascularization, cell-to-cell contact, or cell-to-cell communication. The composition is suitable for treating injured cardiac tissue that has a deficiency in one or more of cardiac output, cardiac tissue viability, cardiac blood flow according to several embodiments. In some embodiments, the positive effector is selected from the group consisting of one or more of the following: periostin, thymosin beta-4, hepatocyte growth factor, insulin-like growth factor, fibroblast growth factor, and a transcription factor, the negative effector is selected from the group consisting of one or more of the following: adenosine, an adenosine agonist, an adenosine receptor agonist, a phosphoinositide 3-kinase inhibitor, a caspase inhibitor, cyclosporine, an opiod receptor antagonist, pinacidil, a nitric oxide donor, poly(ADP-ribose) inhibitors, sodium-hydrogen exchange inhibitors, and thymosin beta-4, and the ancillary effector is selected from the group consisting of one or more of the following: p38 MAP kinase inhibitors, phosphodiesterase inhibitors, stem cell factor, and transforming growth factor beta.

In several embodiments, compositions for improving survival, engraftment and/or proliferation of stem cells are provided, wherein said compositions comprise effectors such as thymosin beta-4, adenosine, and ISL-1, and optionally stem cells (such as non-embryonic cardiac stem cells). The use of those compositions in the preparation a medicament for treating cardiac tissue is provided in several embodiments.

### TERMINOLOGY

The term “about” or “approximately” means within 20%, preferably within 10%, and more preferably within 5% (or 1% or less) of a given value or range.

As used herein, “administer” or “administration” shall be given their ordinary meaning and shall refer to the act of injecting or otherwise physically delivering a substance as it exists outside the body (e.g., an effector provided herein) into a patient, such as by, but not limited to, intramyocardial, pulmonary (e.g., inhalation), mucosal (e.g., intranasal), intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptom thereof, is being prevented, administration of the substance typically occurs before the onset of the disease or symptoms thereof.

The term “ancillary effector” shall be given its ordinary meaning and shall refer to a molecule that can be used or administered in conjunction with a positive and/or a negative effector, which contributes to the beneficial treatment of injured cardiac tissue. Exemplary functions of an ancillary effector include, but are not limited to, facilitating the functions of positive and/or negative effectors or acting as an angiogenic agent, or facilitating cell-to-cell interaction or communication. Non-limiting examples of ancillary effectors include p38 MAP kinase inhibitors, phosphodiesterase inhibitors, stem cell factors and transforming growth factor (TGF) (e.g., TGFβ or TGFβ3).

The term “angiogenic agent” as used herein shall be given its ordinary meaning and shall refer to a molecule capable of activating or otherwise promoting angiogenesis. Angiogenesis is a process by which new blood vessels grow and develop.

The term “autologous” as used herein shall be given its ordinary meaning and shall refer to organs, tissues, cells, fluids or other bioactive molecules that are reimplanted in the same individual that they originated from. Non-limiting examples of autologous transplants or grafts include bone, bone marrow, skin biopsy, heart biopsy, cartilage and blood and stem cells, e.g., CDCs.

The term “cardiac cells” as used herein shall be given its ordinary meaning and shall refer to any cells present in the heart that provide a cardiac function, such as heart contraction or blood supply, or otherwise serve to maintain the structure of the heart. Cardiac cells as used herein encompass cells that exist in the epicardium, myocardium or endocardium of the heart. Cardiac cells also include, for example, cardiac muscle cells or cardiomyocytes; cells of the cardiac vasculatures, such as cells of a coronary artery or vein. Other non-limiting examples of cardiac cells include epithelial cells, endothelial cells, fibroblasts, cardiac conducting cells and cardiac pacemaking cells that constitute the cardiac muscle, blood vessels and cardiac cell supporting structure.

The term “cardiac function” shall be given its ordinary meaning and shall refer to the function of the heart, including global and regional functions of the heart. The term “global” cardiac function as used herein shall be given its ordinary meaning and shall refer to function of the heart as a whole. Such function can be measured by, for example, stroke volume, ejection fraction, cardiac output, cardiac contractility, etc. The term “regional cardiac function” shall be given its ordinary meaning and shall refer to the function of a portion or region of the heart. Such regional function can be measured, for example, by wall thickening, wall motion, myocardial mass, segmental shortening, ventricular remodeling, new muscle formation, the percentage of cardiac cell proliferation and programmed cell death, angiogenesis and the size of fibrous and infarct tissue. In certain embodiments, cardiac cell proliferation is assessed by the increase in the nuclei or DNA synthesis of cardiac cells, cell cycle activities or cytokinesis. In certain embodiments, programmed cell death is measured by TUNEL assay that detects DNA fragmentation. In some embodiments, angiogenesis is detected by the increase in arteriolar and/or capillary densities. Techniques for assessing global and regional cardiac function are known in the art. For example, techniques that can be used to measure regional and global cardiac function include, but are not limited to, echocardiography (e.g., transthoracic echocardiogram, transesophageal echocardiogram or 3D echocardiography), cardiac angiography and hemodynamics, radionuclide imaging, magnetic resonance imaging (MRI), sonomicrometry and histological techniques.

The term “cardiac tissue” as used herein shall be given its ordinary meaning and shall refer to tissue of the heart, for example, the epicardium, myocardium or endocardium, or portion thereof, of the heart. The term “injured” cardiac tissue as used herein shall be given its ordinary meaning and shall refer to a cardiac tissue that is, for example, ischemic, infarcted, reperfused, or otherwise focally or diffusely injured or diseased. Injuries associated with a cardiac tissue include any areas of abnormal tissue in the heart, including any areas caused by a disease, disorder or injury and includes damage to the epicardium, endocardium and/or myocardium. Non-limiting examples of causes of cardiac tissue injuries include acute or chronic stress (e.g., systemic hypertension, pulmonary hypertension or valve dysfunction), atheromatous disorders of blood vessels (e.g., coronary artery disease), ischemia, infarction, inflammatory disease and cardiomyopathies or myocarditis.

The term “engraftment” as used herein shall be given its ordinary meaning and shall refer to the process by which transplanted stem cells (e.g., autologous stem cells) are accepted by a host tissue, survive and persist in that environment. In certain embodiments, the transplanted stem cells further reproduce.

The terms “generate,” “generation” and “generating” as used herein shall be given their ordinary meaning and shall refer to the production of new cardiac cells in a subject and optionally the further differentiation into mature, functioning cardiac cells. In some embodiments, generation of cardiac cells comprises regeneration of the cardiac cells. In certain embodiments, generation of cardiac cells comprises improving survival, engraftment and/or proliferation of the cardiac cells.

The term “negative effector” shall be given its ordinary meaning and shall refer to a molecule which inhibits or otherwise reduces apoptotic cell death and inflammation related to cardiac tissue injury resulting from, for example, infarction, ischemia or reperfusion. Non-limiting examples of negative effectors include PI3-K inhibitors, caspase inhibitors, cyclosporine, hypoxia inducible factors, delta opioids, pinacidil, poly(ADP-ribose) polymerase inhibitors, nitric oxide donors, fibrin-derived peptides, Na—H exchange inhibitors, adenosine, adenosine agonists, an adenosine receptor agonists, thymosin (e.g., a beta-thymosin, such as thymosin β4) and combinations thereof.

As used herein, the term “peri-infarct zone” shall be given its ordinary meaning and shall refer to area at the junction between the normal tissue and the infarcted tissue, i.e., an area of a dying or dead heart tissue resulting from obstruction of blood flow to the heart muscle that results from a relative or absolute insufficiency of blood supply. In certain embodiments of the methods provided herein, the stem cells are administered into the peri-infarct zone of the cardiac tissue. In certain embodiments, the stem cells are administered in the peri-infarct zone together with a positive effector, a negative effector, an ancillary effector or a combination thereof.

The term “positive effector” shall be given its ordinary meaning and shall refer to a molecule capable of activating or otherwise enhancing or promoting cell proliferation, cell engraftment, cell migration, cell differentiation and/or cell cycle re-entry of differentiated cardiac cells. Positive effectors include, for example, embryonic factors, including factors expressed during embryogenesis or during an adult response to tissue injury (e.g., periostin, also known as osteoblast-specific factor), fibroblast growth factors, hepatocyte growth factors, transcription factors (e.g., embryonic transcription factors, such as ISL LIM homeobox 1, ISL-1, Hand1 and Mef2c), insulin-like growth factors, thymosin (e.g., a beta-thymosin, such as thymosin β4) or combinations thereof.

As used herein, the terms “preserve,” “preservation of” and “preserving” in the context of injured tissue shall be given their ordinary meaning and shall refer to protection and/or maintenance of the cardiac tissue, or the functions thereof, such that the tissue is not further injured or compromised, or that the rate of further injury or compromise is slowed relative to the rate in the absence of the intervention at issue. In certain embodiments, preserving injured cardiac tissue comprises prevention or reduction of apoptosis of cells (e.g., cardiomyocytes or stem cells). In certain embodiments, preserving injured cardiac tissue comprises prevention or reduction of cell inflammation.

The terms “regenerate,” “regeneration” and “regenerating” as used herein in the context of injured tissue shall be given their ordinary meaning and shall refer to the process of growing and/or developing new cardiac tissue in a heart or cardiac tissue that has been injured, for example, injured due to ischemia, infarction, reperfusion, or other disease. In certain embodiments, cardiac tissue regeneration comprises activation and/or enhancement of cell proliferation. In certain embodiments, cardiac tissue regeneration comprises activation and/or enhancement of cell migration.

The term “stem cells” shall be given its ordinary meaning and shall refer to cells that have the capacity to self-renew and to generate differentiated progeny. The term “pluripotent stem cells” shall be given its ordinary meaning and shall refer to stem cells that has complete differentiation versatility, i.e., the capacity to grow into any of the fetal or adult mammalian body's approximately 260 cell types. For example, pluripotent stem cells have the potential to differentiate into three germ layers: endoderm (e.g., blood vessels), mesoderm (e.g., muscle, bone and blood) and ectoderm (e.g., epidermal tissues and nervous system), and therefore, can give rise to any fetal or adult cell type. The term “induced pluripotent stem cells” shall be given its ordinary meaning and shall refer to differentiated mammalian somatic cells (e.g., adult somatic cells, such as skin) that have been reprogrammed to exhibit at least one characteristic of pluripotency (see, e.g., co-owned U.S. Application No. 61/116,623, filed Nov. 20, 2008, which is herein incorporated by reference in its entirety). The term “multipotent stem cells” shall be given its ordinary meaning and shall refer to a stem cell that has the capacity to grow into any subset of the fetal or adult mammalian body's approximately 260 cell types. For example, certain multipotent stem cells can differentiate into at least one cell type of ectoderm, mesoderm and endoderm germ layers. The term “embryonic stem cells” shall be given its ordinary meaning and shall refer to stem cells derived from the inner cell mass of an early stage embryo, e.g., human, that can proliferate in vitro in an undifferentiated state and are pluripotent. The term “cardiac stem cells” shall be given its ordinary meaning and shall refer to stem cells obtained from or derived from cardiac tissue. The term “cardiosphere-derived cells (CDCs)” as used herein shall be given its ordinary meaning and shall refer to undifferentiated cells that grow as self-adherent clusters from subcultures of postnatal cardiac surgical biopsy specimens. CDCs can express stem cell as well as endothelial progenitor cell markers, and typically possess properties of adult cardiac stem cells. For example, human CDCs can be distinguished from human cardiac stem cells in that human CDCs typically do not express multidrug resistance protein 1 (MDR1; also known as ABCB1), CD45 and CD133 (also known as PROM1). See, e.g., Passier et al. (2008) Nature 453:322. CDCs are capable of long-term self-renewal, and can differentiate in vitro to yield cardiomyocytes or vascular cells after ectopic (dorsal subcutaneous connective tissue) or orthotopic (myocardial infarction) transplantation in SCID beige mouse. See also U.S. Pub. No. 2008/0267921, which is herein incorporated by reference in its entirety. The term “bone marrow stem cells” shall be given its ordinary meaning and shall refer to stem cells obtained from or derived from bone marrow. The term “placenta-derived stem cells” or “placental stem cells” shall be given their ordinary meaning and shall refer to stem cells obtained from or derived from a mammalian placenta, or a portion thereof (e.g., amnion or chorion). The term “amniotic stem cells” shall be given its ordinary meaning and shall refer to stem cells collected from amniotic fluid or amniotic membrane. The term “embryonic germ cells” shall be given its ordinary meaning and shall refer to cells derived from primordial germ cells, which exhibit an embryonic pluripotent cell phenotype. The term “spermatocytes” shall be given its ordinary meaning and shall refer to male gametocytes derived from a spermatogonium.

As used herein, the terms “subject” and “patient” shall be given their ordinary meaning and are used interchangeably. As used herein, a subject is a mammal such as a non-primate (e.g., cows, pigs, horses, cats, dogs, rats, rabbits, etc.) or a primate (e.g., monkey and human) having an injured cardiac tissue. In specific embodiments, the subject is a human. In one embodiment, the subject is a mammal with acute heart failure. In another embodiment, the subject is a mammal with chronic heart failure.

The term “synergistic” as used herein shall be given its ordinary meaning and shall refer to a combination of, for example, stems cells, and one or more effectors, which is more effective than the additive effects of any two or more single agents (e.g., stem cells and one effector; or two effectors without stem cells).

As used herein, the terms “treat,” “treatment” and “treating” shall be given their ordinary meaning and shall refer to the reduction or amelioration of the progression, severity, and/or duration of a cardiac tissue injury or a symptom thereof. Treatment as used herein includes, but are not limited to, preserving the injured cardiac tissue, regenerating new cardiac tissue, increasing blood flow to the injured tissue, increasing myocardial perfusion, improving global cardiac function (e.g., stroke volume, ejection fraction, and cardiac output) and regional cardiac function (e.g., ventricular wall thickening, segmental shortening and heart pumping).

### DETAILED DESCRIPTION

Provided herein are methods and compositions for improving the therapeutic benefit of stem cells in the treatment of cardiac injuries. For example, presented herein are improved methods for use of stem cells to regenerate myocardium following a myocardial infarction. Without wishing to be bound by any particular mechanism or theory, it is thought that the methods presented herein provide or modify local cell environment in a manner that provides a beneficial adjunct to use of stem cells for treatment of injured cardiac tissue.

As such, provided herein, for example, are methods for improving survival, engraftment, and proliferation of stem cells in a cardiac tissue. In addition, the methods provided herein are applicable for generating cardiac cells and for treating an injured cardiac tissue in a subject. Moreover, compositions for generating cardiac cells in a subject are also provided herein.

### Stem Cells

In certain embodiments, stem cells useful for the compositions and methods provided herein include those listed in Table 1, and include, for example, embryonic stem cells, amniotic stem cells, bone marrow stem cells, placenta-derived stem cells, embryonic germ cells, cardiac stem cells, CDCs, induced pluripotent stem cells, mesenchymal stem cells, endothelial progenitor cells, and spermatocytes. The stem cells employed can be autologous or heterologous to the subject being treated. In specific embodiments, the stem cells are autologous stem cells.

TABLE 1

| Cell Type | Representative Source |
|---|---|
| Embryonic stem cells | Embryo |
| Amniotic stem cells | Placenta |
| Mesenchymal stem cells | Marrow, fat |
| Endothelial progenitor cells | Marrow, blood |
| Cardiac stem cells | Cardiac biopsy |
| Cardiosphere-derived stem cells | Cardiac biopsy |
| Skeletal myoblast | Muscle biopsy |
| Adult spermatocytes | Testicular biopsy |
| Induced pluripotent stem cells | Skin |

The stem cells can be a homogeneous composition or a mixed cell population, for example, enriched with a particular type of stem cell. Homogeneous cell compositions can be obtained, for example, by cell surface markers characteristic of stem cells, or particular types of stem cells, in conjunction with monoclonal antibodies directed to the specific cell surface markers. Homogenous cell compositions, for example, those comprising cardiosphere-derived cells (CDCs), can also be obtained without the use of antibody reagents for selection using standard techniques (see, e.g., Smith et al. (2007) Circulation 115:896).

In specific embodiments, the stem cells are CDCs. The cells that form the cardiospheres can, for example, be obtained from cardiac surgical biopsy specimens taken from a subject, such as a human (e.g., a human with acute or chronic heart failure or other cardiac injury). In some embodiments, the specimen samples are obtained by a non-invasive method, for instance, by a simple percutaneous entry. The cardiospheres can be disaggregated using standard means known in the art for separating cell clumps or aggregates, for example, agitation, shaking, blending. In some embodiments, the cardiospheres are disaggregated to single cells. In other embodiments, the cardiospheres are disaggregated to smaller aggregates of cells. After disaggregation, the cells can be grown on a solid surface (e.g., glass or plastic), such as a culture dish, a vessel wall or bottom, a microtiter dish, a bead, flask, or roller bottle. The cells can adhere to the material of the solid surface or the solid surface can be coated with a substance that encourages adherence. Such substances are well known in the art and include, for example, fibronectin. hydrogels, polymers, laminin, serum, collagen, gelatin, and poly-L-lysine. In certain embodiments, growth on the surface will be monolayer growth.

After surface growth, the disaggregated cells can be grown under conditions which favor formation of cardiospheres. Repeated cycling between surface growth and suspension growth (cardiospheres) leads to a rapid and exponential expansion of desired cells. The cardiosphere phase can alternatively be eliminated, and instead the cells can be surface expanded, e.g., repeatedly surface expanded, without the formation of cardiospheres. The culturing of CDCs, whether on cell surfaces or in cardiospheres, can be performed in the absence of exogenous growth factors. While fetal bovine serum can be used, other factors have been found to be expendable, such as EGF, bFGF, cardiotrophin-1, and thrombin. More information regarding the preparation and culture of CDCs can be found, for example, in U.S. Pub. No. 2008/0267921, which is incorporated herein by reference in its entirety.

The stem cells can be obtained or derived from any of a variety of sources. For example, subjects that can be the donors (or recipients) of stem cells in the methods and compositions presented herein include, for example, mammals, such as non-primates (e.g., cows, pigs, horses, cats, dogs, rats or rabbits) or primates (e.g., monkeys or humans). In specific embodiments, the subject is a human. In one embodiment, the subject is a mammal, e.g., a human, such as a human with acute or chronic heart failure or other cardiac tissue injury.

While a single species can be the donor by providing the cells and be the recipient by receiving the cells (i.e., autologous stem cells), in some embodiments the donor and recipient of the stem cells may be of different species (i.e., xenogeneic). For instance, porcine cells can be administered into human cardiac tissue. In certain embodiments, the stem cells are allogeneic or syngeneic. In specific embodiments, the stem cells are autologous to the cardiac tissue. Having an autologous source of stem cells from the same individual further decreases the possibility of avoiding transplant rejection such as Graft-versus-Host Disease (GVHD). In certain embodiments, the autologous stem cells are derived from adult non-cardiac tissue. In some embodiments, the stem cells are induced pluripotent stem cells derived or created from somatic adult cells, e.g., dermal fibroblasts, using techniques known in the art (see, e.g., Takahashi et al. (2007) Cell 131:861; Yu et al. (2007) Science 318:1917).

### Effectors

### Positive Effectors

Positive effectors useful in the methods and compositions provided herein can be any molecule that activates, enhances or promotes cardiac cell proliferation, cell engraftment, cell migration, cell differentiation and/or cell cycle re-entry. In certain embodiments, the positive effector has mitogenic effects, for example, that encourage cells to commence cell division, stimulate cell growth, and/or cause other morphogenic effects. In other embodiments, the positive effector induces proliferation by initiating signal transduction pathways leading to cell growth and proliferation, such as integrins, ERK1/2 and/or the PI3-kinase/Akt pathways.

In the instances where the positive effectors are peptides, polypeptides or proteins, the positive effectors provided herein can comprise the entire amino acid sequence, or alternatively a biologically active fragment thereof. The positive effector can be chemically synthesized or purified from a cell, e.g., a prokaryotic, eukaryotic or other cell. In certain embodiments, the positive effector is naturally occurring. In other embodiments, the positive effector is recombinantly produced. In a specific embodiment, the positive effector is or human origin or has a human sequence.

In some embodiments, the positive effector is encoded by a gene that is genetically engineered into the stem (or other) cell using methods known in the art (see, e.g., Sambrook et al. (2001) Molecular Cloning: A Laboratory Manual, Cold Spring Harbor Laboratory Press, Cold Spring Harbor, N.Y.; Maniatis et al. (1982) Molecular Cloning: A Laboratory Manual, Cold Spring Harbor Press, Cold Spring Harbor, N.Y.), which positive effector-encoding gene is expressed in the cell. In certain embodiments, the genetically engineered cell secretes the positive effector into the microenvironment the cell.

In one embodiment, the positive effector is an embryonic factor, including factors expressed during embryogenesis or during an adult response to tissue injury.

For example, in some embodiments, the positive effector embryonic factor is periostin. Sequences of periostin are known in the art. Among these, for example, is human periostin having the following 836 amino acid sequence:

| | |
|---|---|
| 1 | mipflpmfsl llllivnpin annhydkila hsrirgrdqg pnvcalggil gtkkkyfstc |
| 61 | knwykksicg qkttvlyecc pgymrmegmk gcpavlpidh vygtlgivga tttqrysdas |
| 121 | klreeiegkg sftyfapsne awdnldsdir rglesnvnve llnalhshmi nkrmltkdlk |
| 181 | ngmiipsmyn nlglfinhyp ngvvtvncar iihgnqiatn gvvhvidrvl tqigtsiqdf |
| 241 | ieaeddlssf raaaitsdil ealgrdghft lfaptneafe klprgvleri mgdkvaseal |
| 301 | mkyhilntlq csesimggav fetlegntie igcdgdsitv ngikmvnkkd ivtnngvihl |
| 361 | idqvlipdsa kqvielagkq qttftdlvaq lglasalrpd geytllapvn nafsddtlsm |
| 421 | dqrllklilq nhilkvkvgl nelyngqile tiggkqlrvf vyrtavcien scmekgskqg |
| 481 | rngaihifre iikpaekslh eklkqdkrfs tflslleaad lkelltqpgd wtlfvptnda |
| 541 | fkgmtseeke ilirdknalq niilyhltpg vfigkgfepg vtnilkttqg skiflkevnd |
| 601 | tllvnelksk esdimttngv ihvvdkllyp adtpvgndql leilnkliky iqikfvrgst |
| 661 | fkeipvtvyt tkiitkvvep kikviegslq piiktegptl tkvkiegepe frlikegeti |
| 721 | tevihgepii kkytkiidgv pveiteketr eeriitgpei kytristggg eteetlkkll |
| 781 | qeevtkvtkf ieggdghlfe deeikrllqg dtpvrklqan kkvqgsrrrl regrsq |

(SEQ ID NO:1) (NCBI/GenBank Protein Accession No. NP006466; gi209862907) (see, e.g., Takeshita et al. (1993) Biochem. J. 294:271) In certain embodiments, periostin is administered alone or with other positive, negative or ancillary effectors. In certain embodiments, periostin is administered with one or more of the integrin subunits in addition to other positive, negative or ancillary effectors.

In other embodiments, the positive effector is thymosin. In specific embodiments, the thymosin is a beta-thymosin, such as thymosin β4. Sequences of human thymosin β4 are well known. For example, in certain embodiments, human thymosin β4 has the following 44 amino acid sequence:

1 msdkpdmaei ekfdksklkk tetqeknplp sketieqekq ages

(SEQ ID NO:2) (NCBI/GenBank Protein Accession No. NP066932; gi1105606) (see, e.g., Gomez-Marquez et al. (1987) J. Immunol. 143:2740). Thymosin β4, as used herein, also includes thymosin β4 isoforms, as well as thymosin β4 analogues or derivatives, including oxidized thymosin β4, thymosin β4 sulfoxide, N-terminal variants of thymosin β4, C-terminal variants of thymosin β4 and antagonists of thymosin β4. Many thymosin β4 isoforms have been identified and have about 70%, or about 75%, or about 80% or more homology to the known amino acid sequence of thymosin β4. Such isoforms include, for example, thymosin β4a1a, thymosin β9, thymosin β10, thymosin β11, thymosin β12, thymosin β13, thymosin β14 and thymosin β15. These isoforms, along with thymosin β4, generally share a conserved amino acid sequence, LKKTET (SEQ ID NO:3).

In some embodiments, the positive effector is a hepatocyte growth factor (HGF; also known as hepapoietin A and scatter factor) (see, e.g., Nakamura et al. (1992) Prog. Growth Factor Res. 3:67). HGF is secreted as a single inactive polypeptide and is cleaved by serine proteases into a 69-kDa alpha-chain and 34-kDa beta-chain. A disulfide bond between the alpha and beta chains produces the active, heterodimeric molecule. Sequences of HGF are well known. For example, in certain embodiments, human HGF has the following 728 amino acid sequence:

| | |
|---|---|
| 1 | mwvtkllpal llqhvllhll llpiaipyae gqrkrrntih efkksakttl ikidpalkik |
| 61 | tkkvntadqc anrctrnkgl pftckafvfd karkqclwfp fnsmssgvkk efghefdlye |
| 121 | nkdyirncii gkgrsykgtv sitksgikcq pwssmipheh sflpssyrgk dlgenycrnp |
| 181 | rgeeggpwcf tsnpevryev cdipqcseve cmtcngesyr glmdhtesgk icqrwdhqtp |
| 241 | hrhkflpery pdkgfddnyc rnpdgqprpw cytldphtrw eycaiktcad ntmndtdvpl |
| 301 | etteciqgqg egyrgtvnti wngipcqrwd sqyphehdmt penfkckdlr enycrnpdgs |
| 361 | espwcfttdp nirvgycsqi pncdmshgqd cyrgngknym gnlsqtrsgl tcsmwdknme |
| 421 | dlhrhifwep dasklnenyc rnpdddahgp wcytgnplip wdycpisrce gdttptivnl |
| 481 | dhpviscakt kqlrvvngip trtnigwmvs lryrnkhicg gslikeswvl tarqcfpsrd |
| 541 | lkdyeawlgi hdvhgrgdek ckqvlnvsql vygpegsdlv lmklarpavl ddfvstidlp |
| 601 | nygctipekt scsvygwgyt glinydgllr vahlyimgne kcsqhhrgkv tlneseicag |
| 661 | aekigsgpce gdyggplvce qhkmrmvlgv ivpgrgcaip nrpgifvrva yyakwihkii |
| 721 | ltykvpqs |

In certain embodiments, the positive effector is an insulin-like growth factor (IGF, also called somatomedin) (e.g., IGF1 or IGF2). Structurally, both IGF1 and IGF2 resemble insulin and have two chains (A and B) connected by disulfide bonds. Sequences of IGFs are known. Certain human IGF1 and IGF2 are 70 and 67 amino acids in length, respectively. Three main IGFs have been characterized: IGF1 (somatomedin C), IGF2 (somatomedin A), and somatomedin B (see, e.g., Rosenfeld (2003) N. Engl. J. Med. 349:2184. Among these, for example, are at least two isoforms of human IGF1 precursors: IGF 1B (195 amino acids in length):

| | |
|---|---|
| 1 | mgkisslptq lfkccfcdfl kvkmhtmsss hlfylalcll tftssatagp etlcgaelvd |
| 61 | alqfvcgdrg fyfnkptgyg sssrrapqtg ivdeccfrsc dlrrlemyca plkpaksars |
| 121 | vraqrhtdmp ktqkyqppst nkntksqrrk gwpkthpgge qkegteaslq irgkkkeqrr |
| 181 | eigsrnaecr gkkgk |

(SEQ ID NO:5) (NCBI/GenBank Protein Accession No. AAA52537; gi183109), and IGF1A (153 amino acids in length)

| | |
|---|---|
| 1 | mgkisslptq lfkccfcdfl kvkmhtmsss hlfylalcll tftssatagp etlcgaelvd |
| 61 | alqfvcgdrg fyfnkptgyg sssrrapqtg ivdeccfrsc dlrrlemyca plkpaksars |
| 121 | vraqrhtdmp ktqkevhlkn asrgsagnkn yrm |

(SEQ ID NO:6) (NCBI/GenBank Protein Accession No. AAA52538; gi183110) (Rotwein et al. (1986) J. Biol. Chem. 261:4828).

Sequences of IGF2 are also well known. In certain embodiments, the IGF2 is a human IGF2 precursor (isoform 1) having the following 180 amino acid sequence

| | |
|---|---|
| 1 | mgipmgksml vlltflafas cciaayrpse tlcggelvdt lqfvcgdrgf yfsrpasrvs |
| 61 | rrsrgiveec cfrscdlall etycatpaks erdvstpptv lpdnfprypv gkffqydtwk |
| 121 | qstqrlrrgl pallrarrgh vlakeleafr eakrhrplia lptqdpahgg appemasnrk |

(SEQ ID NO:7) (NCBI/GenBank Protein Accession No. NP001007140; gi108796063), and in other embodiments, the IGF2 is a human IGF2 precursor (isoform 2) having the following 236 amino acid sequence:

| | |
|---|---|
| 1 | mvspdpqiiv vapetelasm qvqrtedgvt iiqifwvgrk gellrrtpvs samqtpmgip |
| 61 | mgksmlvllt flafasccia ayrpsetlcg gelvdtlqfv cgdrgfyfsr pasrvsrrsr |
| 121 | giveeccfrs cdlalletyc atpakserdv stpptvlpdn fprypvgkff qydtwkqstq |
| 181 | rlrrglpall rarrghvlak eleafreakr hrplialptq dpahggappe masnrk |

(SEQ ID NO:8) (NCBI/GenBank Protein Accession No. NP002221070; gi189083846) (see, e.g., Shen et al. (1988) Proc. Natl. Acad. Sci. USA 85:1947; Rinderknecht (1978) FEBS Lett. 89:283). In other embodiments, human IGF2 has the following amino acid sequence (52 amino acids):

| | |
|---|---|
| 1 | mgipmgksml vlltflafas cciaayrpse tlcggelvdt |
| | lqfvcgdrgf yf |

(SEQ ID NO:9) (NCBI/GenBank Protein Accession No. AAA52536; gi553303) (see, e.g., Gray et al. (1987) DNA 6:283).

In other embodiments, the positive effector is a fibroblast growth factor (FGF). Sequences of FGFs are known. For example, in certain embodiments, a human FGF has the following 64 amino acid sequence:

| | |
|---|---|
| 1 | qtpneeclfl erleenhynt yiskkhaekn wfvglkkngs ckrgprthyg qkailflplp |
| 61 | vssd |

(SEQ ID NO:10) (NCBI/GenBank Protein Accession No. CAA41788; gi1335059) (see, e.g., Wang et al. (1991) Oncogene) 6:1521). At least 23 members of the FGF family are known to exist (FGF1-FGF23), and any may be used in the methods and compositions provided herein.

In some embodiments, the positive effector is a transcription factor, such as, an embryonic transcription factor in the LIM/homeodomain family of transcription factors (e.g., islet 1 (ISL-1)). Sequences of ISL1 are known (see, e.g., Roose et al., (1999) Genomics 57:301; Karlsson et al., (1990) Nature 344:879. A representative 349 amino acid sequence of human ISL1 is provided below:

| | |
|---|---|
| 1 | mgdmgdppkk krlislcvgc gnqihdqyil rvspdlewha aclkcaecnq yldesctcfv |
| 61 | rdgktyckrd yirlygikca kcsigfsknd fvmrarskvy hiecfrcvac srqlipgdef |
| 121 | alredglfcr adhdvveras lgagdplspl hparplqmaa episarqpal rphvhkqpek |
| 181 | ttrvrtvlne kqlhtlrtcy aanprpdalm keqlvemtgl sprvirvwfq nkrckdkkrs |
| 241 | immkqlqqqq pndktniqgm tgtpmvaasp erhdgglqan pvevqsyqpp wkvlsdfalq |
| 301 | sdidqpafqq lvnfseggpg snstgsevas mssqlpdtpn smvaspiea |

(SEQ ID NO:11) (NCBI/GenBank Protein Accession No. NP002193; gi115387114) (see, e.g., Wang et al. (1994) Endocrinol. 134:1416; Dong et al. (1991) Mol. Endocrinol. 5:1633).

### Negative Effectors

Negative effectors useful in the compositions and methods provided herein can be any molecule that inhibits or otherwise reduces apoptotic cell death and/or inflammation. In certain embodiments, the negative effector reduces the apoptotic cell death or inflammation caused by a cardiac tissue injury (e.g., infarction, ischemia or reperfusion).

In the instances where the negative effectors are peptides, polypeptides or proteins, the negative effectors provided herein can comprise the entire amino acid sequence, or alternatively a biologically active fragment thereof. The negative effector can be chemically synthesized or purified from a cell, e.g., a prokaryotic, eukaryotic or other cell. In certain embodiments, the negative effector is naturally occurring. In other embodiments, the negative effector is recombinantly produced. In a specific embodiment, the negative effector is or human origin or has a human sequence.

In some embodiments, the negative effector is encoded by a gene that is genetically engineered into the stem (or other) cell using methods known in the art (see, e.g., Sambrook et al. (2001) Molecular Cloning: A Laboratory Manual, Cold Spring Harbor Laboratory Press, Cold Spring Harbor, N.Y.; Maniatis et al. (1982) Molecular Cloning: A Laboratory Manual, Cold Spring Harbor Press, Cold Spring Harbor, N.Y.), which negative effector-encoding gene is expressed in the cell. In certain embodiments, the genetically engineered cell secretes the negative effector into the microenvironment the cell.

In certain embodiments, the negative effector is adenosine, an adenosine agonist or an adenosine receptor agonist. These agents can be delivered to the cardiac tissue, for example, by direct injection into the tissue by intracoronary injection, or embedding the agent in an adjacent release system, such as a matrix. Adenosine, as shown below, is a purine nucleoside composed of adenine attached to ribofuranose via a β-N9-glycosidic bond.

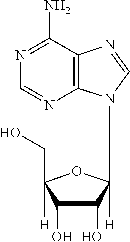

In certain embodiments, the adenosine is used as a 6 or 12 mg bolus dose (Fujisawa Healthcare, Inc.; Deerfield, Ill.), e.g., for intravenous or intramyocardial administration. The anti-inflammatory effect of adenosine is thought to be mediated through its interaction with the A_{2A} receptor. In addition to adenosine, A_{2A} receptor can be activated by other small molecules, termed adenosine receptor agonists. As such, in certain embodiments of the methods and compositions provided herein, the negative effector is an adenosine receptor agonist, e.g., an A_{2A} receptor agonist (see, e.g., Trevethick et al., (2008) Br J Pharmacol. 155:463, which is incorporated herein by reference in its entirety).

In some embodiments, the negative effector is a phosphoinositide 3-kinase (PI3-K) inhibitor, which is a molecule that decrease or otherwise blocks the action of PI3-K. PI3-K inhibitors are known (see, e.g., Redaelli et al. (2006) Mini Rev. Med. Chem. 6:1127; Lindsley et al. (2008) Curr. Cancer Drug Targets 8:7). For example, in certain embodiments, the PI3-K inhibitor is wortmannin or LY294002, exemplary structures of which are shown below, or derivatives thereof.

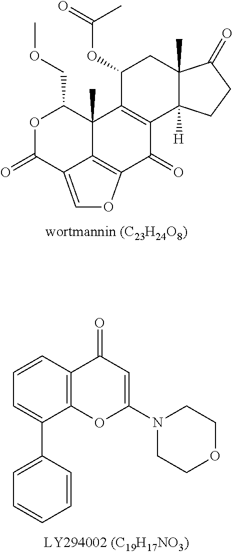

In some embodiments, the negative effector is a caspase inhibitor, which is a molecule that decreases or otherwise blocks the action of caspases, e.g., the initiation of a caspase reaction. Caspase inhibitors can, for example, inhibit any caspase, including any of the at least 14 members of the caspase family, e.g., initiator and effector caspases. Caspase inhibitors are known, and can be designed to include a peptide recognition sequence attached to a functional group such as an aldehyde (CHO), chloromethylketone (CMK), fluoromethylketone (FMK) or fluoroacyloxymethyl ketone (FAOM). The peptide recognition sequence corresponding to that found in endogenous substrates determines the specificity of a particular caspase. Examples and structures of caspase inhibitors are well known (see, e.g., O'Brien et al. (2004) Mini Rev. Med. Chem. 4:153; Ruel (1999) Herz 24:236; Guttenplan et al. (2001) Heart Dis. 3:313). Exemplary caspase inhibitors suitable for methods and compositions presented herein include, but are not limited to, caspase inhibitors (e.g., caspase inhibitor I, II, III, IV, VI, VIII or X); caspase-1 inhibitors (e.g., inhibitors I , II, IV, V or VI); caspase-2 inhibitors (e.g., inhibitors I, II; caspase-3 inhibitors (e.g., inhibitors I, II, III, IV or VII); caspase-3/7 inhibitors (e.g., inhibitors I or II); caspase-4 inhibitors (e.g., inhibitor I); caspase-6 inhibitors (e.g., inhibitors I or II); caspase-8 inhibitors (e.g., inhibitors I or II); caspase-9 inhibitors (e.g., inhibitors I, II or III); and caspase-13 inhibitors (e.g., inhibitors I or II), which are commercially available (e.g., Calbiochem/EMD Biosciences (San Diego, Calif.)).

In certain embodiments, the negative effector is cyclosporine. The structure of cyclosporine is known, and an exemplary structure is shown below.

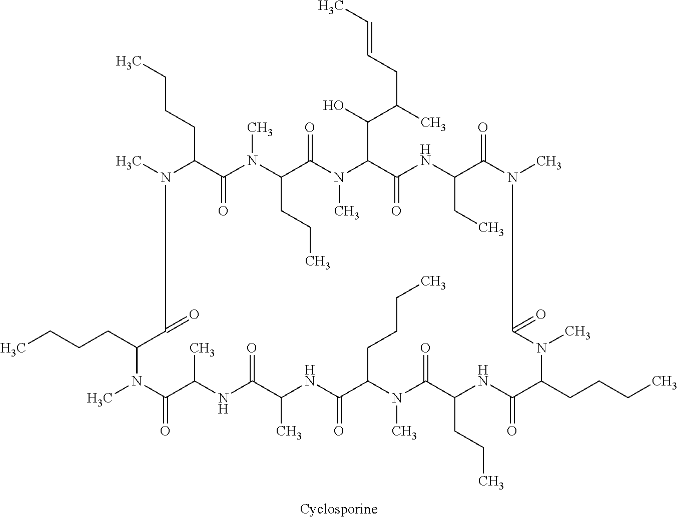

In some embodiments, the negative effector is a hypoxia inducible factor (HIF). Sequences of HIF are known. Among these, in certain embodiments, the human HIF-1 alpha subunit has the following 826 amino acid sequence:

| | |
|---|---|
| 1 | megaggandk kkisserrke ksrdaarsrr skesevfyel ahqlplphnv sshldkasvm |
| 61 | rltisylrvr klldagdldi eddmkaqmnc fylkaldgfv mvltddgdmi yisdnvnkym |
| 121 | gltqfeltgh svfdfthpcd heemremlth rnglvkkgke qntqrsfflr mkctltsrgr |
| 181 | tmniksatwk vlhctghihv ydtnsnqpqc gykkppmtcl vlicepiphp snieipldsk |
| 241 | tflsrhsldm kfsycderit elmgyepeel lgrsiyeyyh aldsdhltkt hhdmftkgqv |
| 301 | ttgqyrmlak rggyvwvetq atviyntkns qpqcivcvny vvsgiiqhdl ifslqqtecv |
| 361 | lkpvessdmk mtqlftkves edtsslfdkl kkepdaltll apaagdtiis ldfgsndtet |
| 421 | ddqqleevpl yndvmlpspn eklqninlam splptaetpk plrssadpal nqevalklep |
| 481 | npeslelsft mpqiqdqtps psdgstrqss pepnspseyc fyvdsdmvne fklelveklf |
| 541 | aedteaknpf stqdtdldle mlapyipmdd dfqlrsfdql splesssasp esaspqstvt |
| 601 | vfqqtqiqep tanattttat tdelktvtkd rmedikilia spspthihke ttsatsspyr |
| 661 | dtqsrtaspn ragkgvieqt ekshprspnv lsvalsqrtt vpeeelnpki lalqnaqrkr |
| 721 | kmehdgslfq avgigtllqq pddhaattsl swkrvkgcks seqngmeqkt iilipsdlac |
| 781 | rllgqsmdes glpqltsydc evnapiqgsr nllqgeellr aldqvn |

(SEQ ID NO:12) (NCBI/GenBank Protein Accession No. AAC50152; gi881346); and HIF-1 beta subunit (also known as aryl hydrocarbon nuclear translocator (ARNT)) having the following 789 amino acid sequence:

| | |
|---|---|
| 1 | maattanpem tsdvpslgpa iasgnsgpgi qgggaivqra ikrrpgldfd ddgegnskfl |
| 61 | rcdddqmsnd kerfarsdde qssadkerla renhseierr rrnkmtayit elsdmvptcs |
| 121 | alarkpdklt ilrmavshmk slrgtgntst dgsykpsflt dqelkhlile aadgflfivs |
| 181 | cetgrvvyvs dsvtpvlnqp qsewfgstly dqvhpddvdk lreqlstsen altgrildlk |
| 241 | tgtvkkegqq ssmrmcmgsr rsficrmrcg sssvdpvsvn rlsfvrnrcr nglgsvkdge |
| 301 | phfvvvhctg yikawppagv slpdddpeag qgskfclvai grlqvtsspn ctdmsnvcqp |
| 361 | tefisrhnie giftfvdhrc vatvgyqpqe llgknivefc hpedqqllrd sfqqvvklkg |
| 421 | qvlsvmfrfr sknqewlwmr tssftfqnpy sdeieyiict ntnvknssqe prptlsntiq |
| 481 | rpqlgptanl plemgsgqla prqqqqqtel dmvpgrdgla synhsqvvqp vtttgpehsk |
| 541 | pleksdglfa qdrdprfsei yhninadqsk gissstvpat qqlfsqgntf pptprpaenf |
| 601 | rnsglappvt ivqpsasagq mlaqisrhsn ptqgatptwt pttrsgfsaq qvatqatakt |
| 661 | rtsqfgvgsf qtpssfssms lpgaptaspg aaaypsltnr gsnfapetgq tagqfqtrta |
| 721 | egvgvwpqwq gqqphhrsss seqhvqqppa qqpgqpevfq emlsmlgdqs nsynneefpd |
| 781 | ltmfppfse |

(SEQ ID NO:13) (NCBI/GenBank Protein Accession No. AAC50152; gi881346) (see, e.g., Wang et al. (1995) Proc. Natl. Acad. Sci. USA 92:5510).

In certain embodiments, the negative effector is delta opioid agonist. Opioid receptor agonists are known (see, e.g., Kaczor et al. (2002) Curr. Med. Chem. 9:1567; Eguchi (2004) Med. Res. Rev. 24:182; Thomas et al. (2001) J. Med. Chem. 44:972). For example, in certain embodiments, the opioid receptor agonist is BW373U86 (see, e.g., Chang et al. (2001) J. Pharmacol. Exp. Ther. 267:852) or DPI-287 (see, e.g., Jutkiewicz et al. (2006) Mol. Interven. 6:162), of which the exemplary structures are shown below.

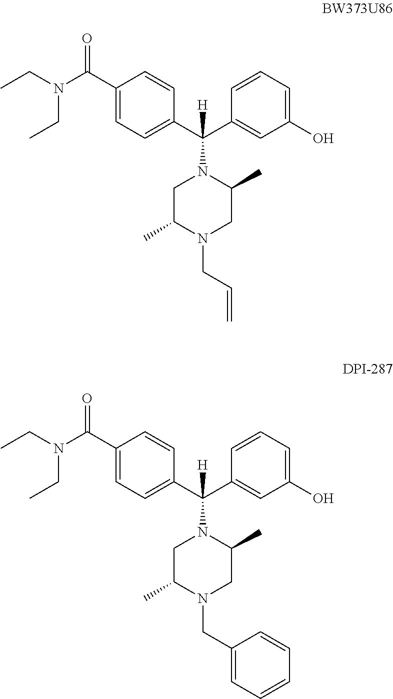

In certain embodiments, the negative effector is pinacidil. The structure of pinacidil is known, and an exemplary structure is shown below.

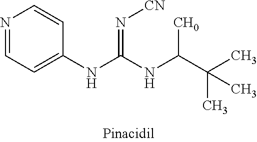

In some embodiments, the negative effector is a nitric oxide donor. As used herein, a nitric oxide donor refers to an agent that contains a nitric oxide moiety and which directly releases or chemically transfers nitrogen monoxide (nitric oxide), for example, in its positively charged nitrosonium form, to another molecule. Nitric oxide donors suitable for methods and compositions provided herein are known, and include, for example, S-nitrosothiols, nitrites, N-oxo-N-nitrosamines, and substrates of various forms of nitric oxide synthase.

Other representative negative effectors that are suitable for methods and compositions provided herein, include, for example, poly(ADP-ribose) polymerase inhibitors, fibrin-derived peptide or Na—H exchange inhibitors. As discussed above, certain positive effectors (e.g., thymosin β4) also exhibit characteristics of negative factors, and thus belong to both groups. For example, thymosin β4 is both a positive effector and a negative effector, as these terms are used herein.

### Ancillary Effectors

The ancillary effectors that can be used in the methods and compositions provided herein can be any molecule used or administered in conjunction with a positive and/or a negative effector, which contributes to the beneficial treatment of injured cardiac tissue. In some embodiments, the ancillary effector facilitates the functions of positive and/or negative effectors. In certain embodiments, the ancillary effector promotes angiogenesis (e.g., as an angiogenic agent) or revascularization. In other embodiments, the ancillary effector facilitates cell-to-cell interaction (e.g., contact or attachment of cells to one another) or communication.

In the instances where the ancillary effectors are peptides, polypeptides or proteins, the ancillary effectors provided herein can comprise the entire amino acid sequence, or alternatively a biologically active fragment thereof. The ancillary effector can be chemically synthesized or purified from a cell, e.g., a prokaryotic, eukaryotic or other cell. In certain embodiments, the ancillary effector is naturally occurring. In other embodiments, the ancillary effector is recombinantly produced. In a specific embodiment, the ancillary effector is or human origin or has a human sequence.

In some embodiments, the ancillary effector is encoded by a gene that is genetically engineered into the stem (or other) cell using methods known in the art (see, e.g., Sambrook et al. (2001) Molecular Cloning: A Laboratory Manual, Cold Spring Harbor Laboratory Press, Cold Spring Harbor, N.Y.; Maniatis et al. (1982) Molecular Cloning: A Laboratory Manual, Cold Spring Harbor Press, Cold Spring Harbor, N.Y.), which ancillary effector-encoding gene is expressed in the cell. In certain embodiments, the genetically engineered cell secretes the ancillary effector into the microenvironment the cell.

In certain embodiments, the ancillary effector is a p38 MAP kinase inhibitor. p38 kinase is proline-directed serine/threonine kinase of the mitogen-activated protein kinase (MAPK) family. Small molecule p38 inhibitors are known and commercially available, for example, RWJ-67657, SB203580, SB202190, SB239063, BIRB796 and VX-745. An exemplary structure of SB20583 is provided below.

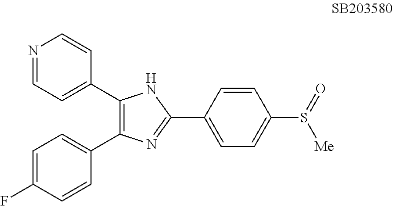

In some embodiments, the ancillary effector is a phosphodiesterase (PDE) inhibitor. In mammals, PDEs include 11 family members, such as PDE1, PDE2, PDE3, PDE4 and PDE5. PDE inhibitors are known. For example, PDE1 inhibitors include vinpocetine; PDE2 inhibitors include erythro-9-(2-hydroxy-3 nonyl)adenine; PDE3 inhibitors include cilostazol, milrinone, enoximone, and pimobendan; PDE4 inhibitors include mesembrine, rolipram, ibudilast and pentoxifylline; and PDE5 inhibitors include sildenafil, tadalafil, vardenafil, udenafil, avanafil and dipyridanole. An exemplary structures of milrinone are provided below.

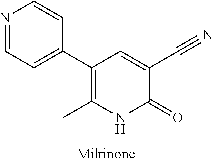

In certain embodiments, the ancillary effector is stem cell factor (SCF). Sequences of SCF are known. Among these, in certain embodiments, human SCF has the following 37 amino acid sequence:

| | |
|---|---|
| 1 | mdvleicsll igltaykels lpkrketcra iqhprkd |

(SEQ ID NO:14) (NCBI/GenBank Protein Accession No. AAB35922; gi1246100) (See, e.g., Sharkey et al. (1995) Biol. Reprod. 53:974).

In some embodiments, the ancillary effector is a transforming growth factor-beta (TGFβ). TGFβ exists in at least three known subtypes in humans, TGFβ1, TGFβ2, and TGFβ3, sequences of which are known. For example, in one embodiment, human TGFβ1 has the following 390 amino acid sequence:

| | |
|---|---|
| 1 | mppsglrllp lllpllwllv ltpgrpaagl stcktidmel vkrkrieair gqilsklrla |
| 61 | sppsqgevpp gplpeavlal ynstrdrvag esaepepepe adyyakevtr vlmvethnei |
| 121 | ydkfkqsths iymffntsel reavpepvll sraelrllrl klkveqhvel yqkysnnswr |
| 181 | ylsnrllaps dspewlsfdv tgvvrqwlsr ggeiegfrls ahcscdsrdn tlqvdingft |
| 241 | tgrrgdlati hgmnrpflll matpleraqh lqssrhrral dtnycfsste knccvrqlyi |
| 301 | dfrkdlgwkw ihepkgyhan fclgpcpyiw sldtqyskvl alynqhnpga saapccvpqa |
| 361 | leplpivyyv grkpkveqls nmivrsckcs |

(SEQ ID NO:15) (NCBI/GenBank Protein Accession No. NP000651; gi63025222) (See, e.g., Miyazono et al. (1988) J. Biol. Chem. 263:6407). In other embodiments, the TGFβ is human TGFβ2, having the following 414 amino acid sequence:

| | |
|---|---|
| 1 | mhycvlsafl ilhlvtvals lstcstldmd qfmrkrieai rgqilsklkl tsppedypep |
| 61 | eevppevisi ynstrdllqe kasrraaace rersdeeyya kevykidmpp ffpsenaipp |
| 121 | tfyrpyfriv rfdvsamekn asnlvkaefr vfrlqnpkar vpeqrielyq ilkskdltsp |
| 181 | tqryidskvv ktraegewls fdvtdavhew lhhkdrnlgf kislhcpcct fvpsnnyiip |
| 241 | nkseelearf agidgtstyt sgdqktikst rkknsgktph lllmllpsyr lesqqtnrrk |
| 301 | kraldaaycf rnvqdncclr plyidfkrdl gwkwihepkg ynanfcagac pylwssdtqh |
| 361 | srvlslynti npeasaspcc vsqdleplti lyyigktpki eqlsnmivks ckcs |

(SEQ ID NO:16) (NCBI/GenBank Protein Accession No. AAH99635; gi68563371) (See also, e.g., Webb et al. (1988) DNA 7:493). In yet another embodiment, the TGFβ3 is TGFβ3, which has the following 412 amino acid sequence:

| | |
|---|---|
| 1 | mkmhlqralv vlallnfatv slslstcttl dfghikkkrv eairgqilsk lrltsppept |
| 61 | vmthvpyqvl alynstrell eemhgereeg ctqentesey yakeihkfdm iqglaehnel |
| 121 | avcpkgitsk vfrfnvssve knrtnlfrae frvlrvpnps skrnegriel fqilrpdehi |
| 181 | akqryiggkn lptrgtaewl sfdvtdtvre wllrresnlg leisihcpch tfqpngdile |
| 241 | nihevmeikf kgvdneddhg rgdlgrlkkq kdhhnphlil mmipphrldn pgqggqrkkr |
| 301 | aldtnycfrn leenccvrpl yidfrqdlgw kwvhepkgyy anfcsgpcpy lrsadtthst |
| 361 | vlglyntlnp easaspccvp qdlepltily yvgrtpkveq lsnmvvksck cs |

(SEQ ID NO:17) (NCBI/GenBank Protein Accession No. NP003230; gi4507465) (See also, e.g., Ten Dijke et al. (1988) Proc. Natl. Acad. Sci. USA 85:4715).

### Methods

In several embodiments, there is provided a method for improving survival of stem cells in a cardiac tissue, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the cardiac tissue with the stem cells, such that survival of the stem cells is improved relative to survival of stem cells that have undergone (b) but not (a).

In several embodiments, there is provided a method for engraftment of stem cells in a cardiac tissue, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the cardiac tissue with the stem cells, such that engraftment of the stem cells occurs.

In several embodiments, there is provided a method for improving proliferation of stem cells in a cardiac tissue, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the cardiac tissue with the stem cells, such that proliferation of the stem cells is improved relative to proliferation of stem cells that have undergone (b) but not (a).

In several embodiments, there is provided a method for generating cardiac cells in a subject, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting a cardiac tissue of the subject with the stem cells such that cardiac cells are generated.

In several embodiments, there is provided a method for treating an injured cardiac tissue in a subject, comprising: (a) contacting stem cells with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another; and (b) contacting the injured cardiac tissue with the stem cells, such that the cardiac tissue is treated.

In several embodiments, there is provided a method for treating an injured cardiac tissue in a subject, comprising: (a) contacting the injured cardiac tissue with stem cells; and (b) contacting the injured cardiac tissue with (i) a positive effector and a negative effector, wherein the positive effector is different from the negative effector; (ii) a positive effector and an ancillary effector, wherein the positive effector is different from the ancillary effector; (iii) a negative effector and an ancillary effector, wherein the negative effector is different from the ancillary effector; or (iv) a positive effector, a negative effector and an ancillary effector, wherein the positive effector, the negative effector and the ancillary effector are different from one another, such that the cardiac tissue is treated.

In several embodiments, there is provided a method for treating an injured cardiac tissue in a subject, comprising: contacting the injured cardiac tissue with (a) CDCs; and (b)(i) a positive effector and a negative effector, wherein the positive effector is different from the negative effector; (ii) a positive effector and an ancillary effector, wherein the positive effector is different from the ancillary effector; (iii) a positive effector and an ancillary effector, wherein the positive effector is different from the ancillary effector; or (iv) a positive effector, a negative effector and an ancillary effector, wherein the positive effector, the negative effector and the ancillary effector are different from one another, such that the cardiac tissue is treated.

In several embodiments, there is provided a method for treating an injured cardiac tissue in a subject, comprising: contacting the injured cardiac tissue with CDCs, adenosine, and at least one of thymosin β4 or periostin, such that the cardiac tissue is treated.

In some embodiments, the methods provided herein for treating an injured cardiac tissue in a subject reduces or ameliorates the progression, severity or duration of a cardiac tissue injury or a symptom thereof. In certain embodiments, treatment preserves the injured cardiac tissue and function thereof, such as by preserving or reducing cell apoptosis, or by reducing cell inflammation. In other embodiments, treatment regenerates cardiac tissue, e.g., cardiac muscle or cardiac vasculature. In some embodiments, treatment activates or enhances cell proliferation or cell migration. In certain embodiments, treatment increases blood flow to the injured tissue. In some embodiments, treatment increases myocardial perfusion. In some embodiments, treatment regenerates new cardiac tissue. In certain embodiments, treatment increases cardiac muscle mass.

In some embodiments, treatment improves global cardiac function. In some embodiments, improvements in global cardiac function are measured by, for example, stroke volume, ejection fraction, cardiac contractility and/or cardiac output using any method known in the art. In some embodiments, improving global cardiac function comprises increasing cardiac output. In certain embodiments, improving global cardiac function comprises increasing ejection fraction (i.e., the fraction of blood pumped out of a ventricle with each heart beat) by at least an absolute range of about 5% to about 25%, about 5% to about 10%, about 5% to about 15%; about 5% to about 20%, about 10% to about 15%, about 10% to about 20%, about 10% to about 25%, about 15% to about 20%, about 15% to about 25%, or about 20% to about 25%. Ejection fraction can be assessed by a number of methods known in the art. In some embodiments, the ejection fraction is determined by echocardiography, cardiac MRI, fast scan cardiac computed axial tomography imaging, or ventriculography. In preferred embodiments, the ejection fraction is assessed by echocardiography.

In other embodiments, treatment improves regional cardiac function. In some embodiments, improvements in regional cardiac function are measured by wall thickening, wall motion, myocardial mass, segmental shortening, ventricular remodeling, new muscle formation, the percentage of cardiac cell proliferation and programmed cell death, angiogenesis and/or the size of fibrous and infarct tissue using any method known in the art. In some embodiments, improving regional cardiac function comprises increasing heart pumping. In certain embodiments, cardiac cell proliferation is assessed by the increase in the nuclei or DNA synthesis of cardiac cells, cell cycle activities or cytokinesis. In certain embodiments, programmed cell death is measured by TUNEL assay that detects DNA fragmentation. In some embodiments, angiogenesis is detected by the increase in arteriolar and/or capillary densities. In certain embodiments, cardiac function before and after treatments are assessed by echocardiography (e.g., transthoracic echocardiogram, transesophageal echocardiogram or 3D echocardiography), cardiac catheterization, magnetic resonance imaging (MRI), sonomicrometry or histological techniques. Techniques in assessing cardiac function can be performed using methods and procedures known in the art (see, e.g., Takehara et al., J. Am. Coll. Cardiol. (2008) 52:1858-65; Laflamme et al., Nature Biotechnol. (2007) 25(9): 1015-24).

In some embodiments, improving global cardiac function comprises increasing ejection fraction by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%.

For example, in some embodiments, a patient having a tissue injury, such as a myocardial infarction, will have an ejection fraction of between about 40% to about 55% that will improve to about 66% after being subjected to a method provided herein (e.g., contacting cardiac tissue with CDCs plus a positive, negative and/or ancillary effector. In certain embodiments, ejection fraction improves to about 55-66% (including 56, 57, 58, 59, 60, 61, 62, 63, 64, and 65%), about 55-60%, about 60-65%, or about 58-63%.

In some embodiments, cardiac tissue subjected to the methods provided herein has been injured, for example, due to ischemia, infarction, reperfusion or occlusion. The cardiac tissue can be focally or diffusely injured or diseased. In some embodiments, the cardiac tissue is injured as a result of acute stress, for example, acute heart failure. In other embodiments, the cardiac tissue is injured as a result of chronic stress, for example, chronic heart failure, systemic hypertension, pulmonary hypertension, valve dysfunction, or atheromatous disorders of blood vessels (e.g., coronary artery disease). In some embodiments, the injured cardiac tissue is in the epicardium, endocardium and/or myocardium. In some embodiments, the subject is a mammal, such as a non-primate. In specific embodiments, the subject is a human. In one embodiment, the subject is a human with acute heart failure or chronic heart failure.

Contacting Stem Cells with Effectors

The positive, negative and/or ancillary effectors can be administered to or contacted with the stem cells in any manner known in the art. In certain embodiments, a positive effector, negative effector and/or ancillary effector is exogenously expressed in the stem cells. The expression of effectors can be accomplished, for example, using an expression system by introducing the DNA encoding the desired effectors. Any of the known methods for introducing DNA are suitable, including, but are not limited to, transfection, electroporation, infection using retroviral vectors, lentivirus, adenovirus, or adeno-associated virus vectors (see, e.g., Sambrook et al. (2001) Molecular Cloning: A Laboratory Manual, Cold Spring Harbor Laboratory Press, Cold Spring Harbor, N.Y.; Maniatis et al. (1982) Molecular Cloning: A Laboratory Manual, Cold Spring Harbor Press, Cold Spring Harbor, N.Y.).

In certain embodiments, the effector(s) are added to the medium in which the stem cells are incubated in vitro or ex vivo. The amount of effector(s) added to the tissue culture medium will vary depending on the type of effector being used. Serial dilutions within a range of about three to four orders of magnitude can be used to routinely optimize the conditions using methods known in the art. In certain embodiments, one or more factors is contacted with the stem cells simultaneously for a period of time, e.g., 1, 12 or 24 hours or between about 1 and about 7 days, such as about 1 day, about 2 days, about 3 days, about 4 days, about 5 days, about 6 days, about 7 days, with media supplements as appropriate. In other embodiments, one or more factors is contacted with the stem cells sequentially for a period of time, e.g., 1, 12 or 24 hours or between about 1 and about 7 days, such as about 1 day, about 2 days, about 3 days, about 4 days, about 5 days, about 6 days, about 7 days, with media supplements as appropriate. For example, a positive effector can be added for a period of time and then optionally removed prior to or after the addition of a negative effector.

Contacting Cardiac Tissue with Stem Cells

Contacting cardiac tissue with stem cells that have optionally been pre-treated in vitro or ex vivo with one or more effectors can be accomplished by any of a variety of known methods. For example, cardiac tissue can be contacted via intercoronary infusion of stem cells, for example, CDCs, e.g., autologous CDCs. The stem cells can be delivered systemically or locally to the heart. In certain embodiments, the stem cells are directly injected epicardially into cardiac tissue, for example, during an open chest surgery. In other embodiments, the stem cells are contacted with the cardiac tissue using non-surgical methods, for example, by intravascular (e.g., intracoronary or intravenous) or intramyocardial administration. Stem cells administered to cardiac tissue using non-surgical methods can be prepared in an injectable liquid suspension or any other biocompatible medium. For intravascular approaches, catheters may be advanced through the vasculature and into the heart to inject the cells into the cardiac tissue from within the heart. In one embodiment, the stem cells are contacted with the cardiac tissue by intracoronary administration. In another embodiment, the stem cells are contacted with the cardiac tissue, for example, by intravenous administration, by continuous drip or as a bolus. In yet another embodiment, the stem cells are contacted with the cardiac tissue by intramyocardial administration, for example, using a conventional intracardiac syringe or a controllable endoscopic delivery device, so long as the needle lumen or bore is of sufficient diameter that shear forces will not damage the stem cells. In certain embodiments, the stem cells are contacted with the cardiac tissue using an endocardial approach that delivers materials into the cardiac wall from within the chamber of the heart.

In certain embodiments of the methods provided herein, the stem cells are administered to or contacted with the peri-infarct zone of cardiac tissue. In specific embodiments of the methods provided herein, the stem cells are administered into the peri-infarct zone with a positive effector, a negative effector, an ancillary effector or a combination thereof.

In some embodiments, the stem cells are administered in a system, e.g., long-term, short-term and/or controlled release system, which can improve cell engraftment and persistence. In certain embodiments, the system is a matrix, such as a natural or synthetic matrix (see, e.g., Simpson et al. (2007) Stem Cells 25:2350). The matrix can hold the stem cells in place at the site of injury by serving as scaffolding. This, in turn, can enhance the opportunity for the administered stem cells to proliferate, differentiate and eventually become fully developed cardiomyocytes. As a result of their localization in the myocardial environment, the cells can then integrate with the recipient's surrounding myocardium.

In certain embodiments, the stem cells are administered in a biocompatible medium which is, or becomes a semi-solid or solid matrix in situ at the site of myocardial damage. In some embodiments, the matrix is an injectable liquid which polymerizes to a semi-solid gel at the site of the damaged myocardium, such as collagen and its derivatives, polylactic acid or polygly-colic acid. In other embodiments, the matrix is one or more layers of a flexible, solid matrix that is implanted in its final form, such as impregnated fibrous matrices. The matrix can be, for example, Gelfoam® (Upjohn, Kalamazoo, Mich.) or a biologic matrix. In certain embodiments, the matrix is permanent. In other embodiments, the matrix is degradable or biodegradable. In some embodiments, the stem cells are embedded into a tissue-engineered cardiac patch containing, for example, a collagen matrix. Such a patch can then be attached or otherwise delivered to the cardiac tissue, for example, with a sealant (e.g., fibrin) (see, e.g., Simpson et al. (2007) Stem Cells 25:2350).

In certain embodiments, the stem cells are administered to the cardiac tissue once. In other embodiments, stem cells are administered to cardiac tissue more than one time. In certain embodiments, the stem cells are administered as a cell suspension in a pharmaceutically acceptable liquid medium (e.g., saline or buffer), for example, for systemic administration or local administration directly into the damaged portion of the myocardium. In specific embodiments, administration is localized to the cardiac tissue.

An effective dose of stem cells for use in the methods provided herein will vary depending on the stem cell type used and/or the delivery site (e.g., intracoronary or intramyocardial), and such doses can be readily determined by a physician. In certain embodiments, the number of stem cells, such as CDCs, is in the range of 1×10⁵ to 1×10⁹. For example, cardiac stem cells can be administered in a dose between about 1×10⁶ and 1×10⁸, such as between 1×10⁷ and 5×10⁷. Depending on the size of the damaged region of the heart, more or less cells can be used. A larger region of damage may require a larger dose of cells, and a small region of damage may require a smaller does of cells. On the basis of body weight of the recipient, an effective dose may be between 1×10⁵ and 1×10⁷ per kg of body weight, such as between 1×10⁶ and 5×10⁶ cells per kg of body weight. Patient age, general condition, and immunological status may be used as factors in determining the dose administered, and will be readily determined by the physician.

Contacting Cardiac Tissue with Effectors

In certain embodiments of the methods provided herein, the cardiac tissue is contacted with a positive effector, a negative effector, an ancillary effector or a combination thereof, in addition to being concurrently or sequentially contacted with the stem cells that have optionally been pretreated ex vivo for a period of time with one or more of the same or different effectors. In some embodiments, the cardiac tissue is contacted with the stem cells concurrently with a positive effector, a negative effector, an ancillary effector or a combination thereof. In other embodiments, the cardiac tissue is contacted with the stem cells prior to a positive effector, a negative effector, an ancillary effector or a combination thereof. In still other embodiments, the cardiac tissue is contacted with a positive effector, a negative effector, an ancillary effector or a combination thereof prior to the stem cells. In other embodiments, the cardiac tissue is sequentially contacted first with an effector (e.g., a positive effector), next with the stem cells, and then with a second effector (e.g., a negative and/or ancillary effector).

In certain embodiments, an injured cardiac tissue is contacted with a negative factor prior to the tissue being contacted with stem cells. For example, in such an embodiment, a negative effector, e.g., a factor that reduces inflammation, for example, adenosine, can be contacted with the injured cardiac tissue within 2, 4, 6, 10, 12 or 20 hours, or about 1, about 2, about 3, about 4, about 5, about 6 or about 7 days of the injury, e.g., an infarction. In such embodiments, the injured cardiac tissue is then subsequently contacted with stem cells. In one particular embodiment, such a method comprises contacting with a negative effector at least between about 3 and about 7 days post-injury, and contacting with stem cells about 3, about 4, about 5 or about 6 days post-injury. Without wishing to be bound by any particular mechanism or theory, initial contacting with a negative effector can provide for a post-cardiac injury local inflammatory environment that will increase the therapeutic benefit of contacting the injured cardiac tissue with stem cells.

The positive, negative and/or ancillary effector can be administered to (i.e., contacted with) the cardiac tissue by any of a variety of procedures known in the art either alone or in combination with each other, and optionally in combination with the stem cells. For example, in certain embodiments, cardiac tissue is contacted via intercoronary infusion of an effector combination provided herein, for example, (i) adenosine and tymosin β4, (ii) adenosine and periostin, or (iii) adenosine, thymosin β4 and periostin, either concurrently or sequentially with stem cells (e.g., CDCs) that have been optionally pre-treated ex vivo for a period of time with the same or different combination of effectors. One or more of the effectors, either alone or in combination, and optionally in combination with the stem cells, can be delivered systemically or locally to the heart. In certain embodiments, one or more of the effectors, either alone or in combination, and optionally in combination with the stem cells, are directly injected epicardially into cardiac tissue, for example, during an open chest surgery. In other embodiments, one or more of the effectors, either alone or in combination, and optionally in combination with the stem cells are contacted with the cardiac tissue using non-surgical methods, for example, by intravascular (e.g., intracoronary or intravenous) or intramyocardial administration. One or more of the effectors, either alone or in combination, and optionally in combination with the stem cells, that are administered to cardiac tissue using non-surgical methods can be prepared, for example, in an injectable liquid suspension or any other biocompatible medium. For intravascular approaches, catheters may be advanced through the vasculature and into the heart to inject one or more of the effectors, either alone or in combination, and optionally in combination with the stem cells, into the cardiac tissue from within the heart. In one embodiment, one or more of the effectors, either alone or in combination, and optionally in combination with the stem cells, are contacted with the cardiac tissue by intracoronary administration. In another embodiment, one or more of the effectors, either alone or in combination, and optionally in combination with the stem cells, are contacted with the cardiac tissue, for example, by intravenous administration, by continuous drip or as a bolus. In yet another embodiment, one or more of the effectors, either alone or in combination, and optionally in combination with the stem cells, are contacted with the cardiac tissue by intramyocardial administration, for example, using a conventional intracardiac syringe or a controllable endoscopic delivery device. In certain embodiments, one or more of the effectors, either alone or in combination, and optionally in combination with the stem cells, are contacted with the cardiac tissue using an endocardial approach that delivers the effector(s) and/or stem cells into the cardiac wall from within the chamber of the heart.

In certain embodiments of the methods provided herein, the effectors are administered to or contacted with the peri-infarct zone of cardiac tissue. In specific embodiments of the methods provided herein, the effectors are administered into the peri-infarct zone concurrently or sequentially with stem cells (e.g., CDCs) that have optionally been pre-treated for a period of time with the same or different effector combination ex vivo.

The effector provided herein can be administered to a cardiac tissue by various known methods known in the art, such as by injection (e.g., direct needle injection at the delivery site, subcutaneously or intravenously), oral administration, inhalation, transdermal application, catheter infusion, biolistic injectors, particle accelerators, Gelfoam, other commercially available depot materials, osmotic pumps, oral or suppositorial solid pharmaceutical formulations, decanting or topical applications during surgery, or aerosol delivery. Depending on the route of administration, the composition can be coated with a material to protect the effectors from the action of acids and other natural conditions which can inactivate the effectors. In preferred embodiments, the effectors are administered to the cardiac tissue locally.

In some embodiments, one or more of the effectors, either alone or in combination with each other, are administered in one or more systems, e.g., a long-term, short-term and/or controlled release system(s) that optionally further comprise the stem cells. In one embodiment, the stem cells are provided in a release system with one or more of the effectors. In another embodiment, the stem cells are provided in a release system, but none of the effectors are provided in a release system. In other embodiments, one or more effectors are provided in one or more releases systems (the same or different), but the stem cells are not provided in a release system. In certain embodiments, the system is a matrix, such as a natural or synthetic matrix (see, e.g., Simpson et al. (2007) Stem Cells 25:2350).

In certain embodiments, one or more of the effectors, either alone or in combination with each other, and optionally in combination with the stem cells, are administered in a biocompatible medium which is, or becomes a semi-solid or solid matrix in situ at the site of myocardial damage, such as any of the matrixes described herein. In certain embodiments, one or more of the effectors, either alone or in combination with each other, and optionally in combination with the stem cells, are embedded into a tissue-engineered cardiac patch containing, for example, a collagen matrix. Such a patch can then be attached or otherwise delivered to the cardiac tissue, for example, with a sealant (e.g., fibrin) (see, e.g., Simpson et al. (2007) Stem Cells 25:2350).

In certain embodiments, one or more of the effectors, either alone or in combination with each other, and optionally in combination with the stem cells, are administered to the cardiac tissue once, either concurrently (e.g., effectors and stem cells) or sequentially (e.g., effectors then stem cells, stem cells then effectors, or effector then stem cells, then effectors, for example, within minutes or hours). In other embodiments, one or more of the effectors, either alone or in combination with each other, and optionally in combination with the stem cells, are concurrently or sequentially administered to cardiac tissue more than one time (e.g., several hours, days or months apart).

In some embodiments of the methods provided herein, one or more of the effectors, either alone or in combination with each other, and optionally in combination with the stem cells, are administered to the cardiac tissue of the patient after tissue injury occurs but before or coincident with reperfusion (e.g., after vascular occlusion but before or coincident with angioplasty).

In certain embodiments, one or more of the effectors, either alone or in combination with each other, and optionally in combination with the stem cells, are administered in a pharmaceutically acceptable liquid medium (e.g., saline or buffer), for example, for systemic administration or local administration, e.g., directly into the damaged portion of the myocardium. In specific embodiments, administration is localized to the cardiac tissue.

One or more of the methods of delivery or formulations provided herein can be used to contact the cardiac tissue with one or more of the effectors, either alone or in combination with each other, and the stem cells. For example, in certain embodiments, one or more of the effectors are contacted with the cardiac tissue by a first method of delivery and/or in a first formulation (e.g., direct needle injection of liquid formulation), and the stem cells are concurrently or sequentially contacted with the cardiac tissue by a second method of delivery and/or in a second formulation (e.g., matrix).

In some embodiments, a negative effector (e.g., adenosine) is contacted with the cardiac tissue at the time of tissue injury or shortly thereafter (e.g., within about 1 to 36 hours, such as within about 1 to 6 hours, about 1 to 12 hours, or about 1 to 24 hours). For example, in certain embodiments, the negative effector is administered to a patient having a myocardial infarction, for example, to reduce inflammation, reduce cell apoptosis and/or preserve the cardiac tissue. A period of time later, a heart biopsy can be taken from the patient and CDCs can be derived, cultured and expanded. At the same time, cardiac tissue can be optionally contacted one or more times (concurrently or sequentially) with a positive, negative and/or ancillary factor until administration of the CDCs to the patient a period of time later (e.g., about 1 to 6 months, such as about 1 month, about 2 months, about 3 months, about 4 months, about 5 months, or about 6 months). The CDCs can also be optionally pre-treated with a positive, negative and/or ancillary effector in vitro or ex vivo, e.g., 1 to 3 days, prior to injection into the patient. The patient can then be later injected with one or more doses of (i) the CDCs that have optionally been pretreated with a positive, negative and/or ancillary effector, and (ii) a positive, negative and/or ancillary effector. Finally, the patient can be optionally further treated with a positive, negative and/or ancillary effector for a period of time following initial CDC administration, e.g., every 1, 3, 5 or 7 days for between about 1 and 52 weeks.

In another example, a negative effector (e.g., adenosine) can be contacted with the cardiac tissue, e.g., at the time of balloon angioplasty. A period of time later (e.g., about 5 to 7 days, such as about 5 days, about 6 days or about 7 days), stem cells, such as CDCs, that have optionally been pretreated with a positive, negative and/or ancillary effector, can be administered to the patient along with concurrent or sequential administration of a positive, negative and/or ancillary effector. The patient can then be optionally further treated with a positive, negative and/or ancillary effector for a period of time following initial CDC administration, e.g., every 1, 3, 5 or 7 days for between about 1 and 52 weeks.

In some embodiments, one of the following combinations of stem cells and effectors are contacted with the cardiac tissue by injection into the coronary artery, or alternatively the myocardium, prior to, during or after tissue injury occurs (i) thymosin β4 plus periostin plus stem cells, e.g., CDCs, that have been optionally pre-treated, e.g., for 48 hours, with thymosin β4 and/or periostin, (ii) periostin plus stem cells, e.g., CDCs, that have been optionally pre-treated, e.g., for 48 hours, with periostin, (iii) thymosin β4 plus adenosine plus stem cells, e.g., CDCs, that have been optionally pre-treated, e.g., for 48 hours, with thymosin β4 and/or adenosine; (iv) thymosin β4 plus periostin plus adenosine plus stem cells, e.g., CDCs, that have been optionally pre-treated, e.g., for 48 hours, with thymosin β4, periostin and/or adenosine, or (v) adenosine plus stem cells, e.g., CDCs transfected with a vector comprising a gene encoding ISL-1 that have been optionally pre-treated, e.g., for 48 hours, with adenosine and/or ISL-1, (vi) adenosine and periostin plus stem cells, e.g., CDCs, that have been optionally pre-treated, e.g., for 48-72 hours, with adenosine and/or periostin, or (vii) stem cells, e.g., CDCs transfected with a vector comprising a gene encoding ISL-1 that have been optionally pre-treated, e.g., for 48 hours, with ISL-1. In some embodiments, administration of the stem cells and effector(s) to the patient occurs after tissue injury occurs but before or coincident with reperfusion (e.g., after vascular occlusion but before or coincident with angioplasty).

An effective dose of positive effector, negative effector and/or ancillary effectors that are contacted with stem cells and/or contacted with cardiac tissue will vary depending on the stem cell type used, the delivery site (e.g., intracoronary or intramyocardial), and the patient (e.g., weight) and such doses can be readily determined by a physician (see also, e.g., Physician's Desk Reference, 63^{rd} Ed. (2009) Thomson PDR (Montvale, N.J.)). Patient age, general condition, and immunological status may be used as factors in determining the dose administered, and will be readily determined by the physician.

### Sequence of Administration

The effectors and stem cells used in the methods provided herein can be contacted (or administered) in any order. For example, in one embodiment, the stem cells are contacted with a positive effector and a negative effector concurrently or sequentially (e.g., a positive effector prior to the negative effector or vice versa). In another embodiment, the stem cells are contacted with a positive effector and an ancillary effector concurrently or sequentially (e.g., a positive effector prior to the ancillary effector or vice versa). In one embodiment, the stem cells are contacted with a negative effector and a ancillary effector concurrently or sequentially (e.g., a negative effector prior to the ancillary effector or vice versa). In other embodiments, the stem cells are contacted with a positive effector, a negative effector and an ancillary effector concurrently or sequentially. In certain embodiments, the stem cells are contacted with (i) a positive effector prior to a negative effector and an ancillary effector (ii) a positive effector first, and then concurrently with a negative effector and an ancillary effector, (iii) a positive effector first, followed by a negative effector, and then followed by an ancillary effector, (iv) a positive effector first, followed by an ancillary effector, and then followed by a negative effector (v) a negative effector prior to a positive effector and an ancillary effector, (vi) a negative effector first, and then concurrently with a positive effector and an ancillary effector, (vii) a negative effector first, followed by a positive effector, and then followed by an ancillary effector, (viii) a negative effector first, followed by an ancillary effector, and then followed by a positive effector (ix) an ancillary effector prior to a positive effector and a negative effector, (x) an ancillary effector first, and then concurrently with a positive effector and a negative effector, (xi) an ancillary effector first, followed by a positive effector, and then followed by a negative effector, (xii) an ancillary effector first, followed by a positive effector, and then followed by a negative effector, (xiii) a positive effector and a negative effector concurrently prior to an ancillary effector, (xiv) a positive effector and a negative effector concurrently prior to an ancillary effector, or (xv) a negative effector and an ancillary effector concurrently prior to a positive effector.

Contacting stem cells concurrently or sequentially with a positive, negative and/or ancillary effector can also be done prior to or concurrently with contacting the cardiac tissue of the subject. For example, in some embodiments, (i) the stem cells are contacted with a positive effector and a negative effector prior to contacting the cardiac tissue with the stem cells (e.g., ex vivo), (ii) the stem cells are contacted with a positive effector and a negative effector concurrently with contacting the cardiac tissue with the stem cells, (iii) the stem cells are contacted with a positive effector and an ancillary effector prior to contacting the cardiac tissue with the stem cells (e.g., ex vivo), (iv) the stem cells are contacted with a positive effector and an ancillary effector concurrently with contacting the cardiac tissue with the stem cells, (v) the stem cells are contacted with a negative effector and an ancillary effector prior to contacting the cardiac tissue with the stem cells (e.g., ex vivo), (vi) the stem cells are contacted with a negative effector and an ancillary effector concurrently with contacting the cardiac tissue with the stem cells, (vii) the stem cells are contacted with a positive effector, a negative effector and an ancillary effector prior to contacting the cardiac tissue with the stem cells (e.g. ex vivo), or (viii) the stem cells are contacted with a positive effector, a negative effector and an ancillary effector concurrently with contacting the cardiac tissue with the stem cells.

Contacting the injured cardiac tissue with stem cells can be done prior to or concurrently with contacting the injured cardiac tissue with a positive, negative and/or ancillary effector sequentially or concurrently. For example, in some embodiments, (i) the cardiac tissue is contacted with stem cells prior to contacting the cardiac tissue with a positive effector and a negative effector, (ii) the cardiac tissue is contacted with the stem cells concurrently with contacting the cardiac tissue with a positive effector and a negative effector, (iii) the cardiac tissue is contacted with stem cells prior to contacting the cardiac tissue with a positive effector and an ancillary effector, (iv) the cardiac tissue is contacted with the stem cells concurrently with contacting the cardiac tissue with a positive effector and an ancillary effector, (v) the cardiac tissue is contacted with stem cells prior to contacting the cardiac tissue with a negative effector and an ancillary effector, (vi) the cardiac tissue is contacted with the stem cells concurrently with contacting the cardiac tissue with a negative effector and an ancillary effector, (vii) the cardiac tissue is contacted with stem cells prior to contacting the cardiac tissue with a positive effector, a negative effector and an ancillary effector, or (viii) the cardiac tissue is contacted with the stem cells concurrently with contacting the cardiac tissue with a positive effector, a negative effector and an ancillary effector.

### Compositions

The compositions provided herein, e.g., for generating cardiac cells in a subject, comprise: (a) stem cells, such as CDCs, and (b) two or more of a positive effector, negative effector and ancillary effector, wherein the two or more effectors are different, such that the cardiac tissue is treated. For example, in one embodiment the composition comprises: (a) stem cells, such as CDCs; and (b) a positive effector and a negative effector, wherein the positive effector is different from the negative effector. In another embodiment, the composition for generating cardiac cells in a subject comprises: (a) stem cells, such as CDCs; and (b) a positive effector and an ancillary effector, wherein the positive effector is different from the ancillary effector. In other embodiments, the composition comprises: (a) stem cells, such as CDCs; and (b) a negative effector and an ancillary effector, wherein the negative effector is different from the ancillary effector. In yet other embodiments, the composition comprises: (a) stem cells, such as CDCs; and (b) a positive effector, a negative effector and an ancillary effector, wherein the positive effector, the negative effector and the ancillary effector are different from one another. Any stem cells, positive effector, negative effector, and/or ancillary effector described herein can be used in the compositions.

In specific embodiments, the composition comprises CDCs, adenosine and at least one of thymosin β4 or periostin.

In some embodiments, the composition comprises one of the following combinations of stem cells and effectors: (i) thymosin β4 plus periostin plus stem cells, e.g., CDCs, that have been optionally pre-treated, e.g., for 48-72 hours, with thymosin β4 and/or periostin, (ii) periostin plus stem cells, e.g., CDCs, that have been optionally pre-treated, e.g., for 48-72 hours, with periostin, (iii) thymosin β4 plus adenosine plus stem cells, e.g., CDCs, that have been optionally pre-treated, e.g., for 48-72 hours, with thymosin β4 and/or adenosine; (iv) thymosin β4 plus periostin plus adenosine plus stem cells, e.g., CDCs, that have been optionally pre-treated, e.g., for 48-72 hours, with thymosin β4, periostin and/or adenosine, (v) adenosine plus stem cells, e.g., CDCs transfected with a vector comprising a gene encoding ISL-1 that have been optionally pre-treated, e.g., for 48-72 hours, with adenosine and/or ISL-1, (vi) adenosine and periostin plus stem cells, e.g., CDCs, that have been optionally pre-treated, e.g., for 48-72 hours, with adenosine and/or periostin, or stem cells, e.g., CDCs transfected with a vector comprising a gene encoding ISL-1 that have been optionally pre-treated, e.g., for 48 hours, with ISL-1.

Specific embodiments will be described with reference to the following non-limiting examples, which should be regarded in an illustrative rather than a restrictive sense.

### EXAMPLES

The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

### Example 1

### Expression of Embryologic Transcription Factors in Peri-Infarct Tissue

The expression of embryologic transcription factors in peri-infarct tissue of normal mouse hearts was examined. Myocardial infarction (MI) was created by ligation of the LAD coronary artery in the C57BL/6 mice. Expression of ten known transcription factors that regulate cardiogenesis were determined in normal, peri-infarct and remote myocardial tissue by RT-PCR and Western Blotting at baseline, 2, 7 and 14 days following infarction (n=5 in each group). Immununohistochemistry was used to examine the distribution of transcription factors as well as c-Kit, the stem cell marker, in the myocardial tissue and Masson trichrome to identify collagenous scar. Successful creation of MI was confirmed by upregulation of collagen (>20-fold) and periostin (>90-fold) at 14 days, and by scar formation by histology RNA in situ hybridization on per-infarct tissue and sections was performed using riboprobes specific for Isl1, Mef2c, and Hand1.

Three factors (Isl1, Mef2c, and HAND1) were up-regulated 2- to 5-fold in infarcted myocardium, accompanied by as much as an 18-fold increase in their respective proteins at 14 days post-infarction. Isl1 protein at 14 days was markedly upregulated by Western blot analysis. Immunohistochemistry revealed that Isl1 was co-localized with the stem cell marker c-Kit. Periostin expression increased 90-fold. Without wishing to be bound by any particular mechanism or theory, these data suggest that a response paralleling cardiogenesis is activated after myocardial infarction, but that the balance of local factors favors scar formation rather than tissue regeneration. Thus, the results indicate that the methods presented herein can create or modify a cell environment that favors tissue regeneration.

### Example 2

### Isolation of Cardiac-Derived Stem Cells from Cardiac Biopsy Specimens

Pluripotent stem cells can be isolated from cardiac biopsy specimens or other cardiac tissue using any known methods, for example, the multi-step process described in U.S. Publication No. 2008/0267921, which is incorporated herein by reference in its entirety.

Utilizing such method, cardiac tissue is first obtained via percutaneous endomyocardial biopsy or via sterile dissection of the heart. Once obtained, tissue specimens are stored on ice in a high-potassium cardioplegic solution (containing 5% dextrose, 68.6 mmol/L mannitol, 12.5 meq potassium chloride, and 12.5 meq sodium bicarbonate, with the addition of 10 units/mL of heparin) until they are processed (up to 12 hours later). For processing, specimens are cut into 1-2 mm³ pieces using sterile forceps and scissors; any gross connective tissue is removed. The fragments are then washed with Ca⁺⁺—Mg⁺⁺-free phosphate buffered saline (PBS) and typically digested for 5 min at room temperature with 0.05% trypsin-EDTA. Alternatively the tissue fragments may be digested in type IV collagenase (1 mg/mL) for 30 minutes at 37° C. Preliminary experiments have shown that cellular yield is greater per mg of explant tissue when collagenase is used.

Once digestion is complete, the remaining tissue fragments are washed with “Complete Explant Medium” (CEM) containing 20% heat-inactivated fetal calf serum, 100 Units/mL penicillin G, 100 μg/mL streptomycin, 2 mmol/L L-glutamine, and 0.1 mmol/L 2-mercaptoethanol in Iscove's modified Dulbecco medium to quench the digestion process. The tissue fragments are minced again with sterile forceps and scissors and then transferred to fibronectin-coated (25 μg/mL for at least 1 hour) tissue culture plates, where they are placed, evenly spaced, across the surface of the plate. A minimal amount of CEM is added to the plate, after which it is incubated at 37° C. and 5% CO₂ for 30 minutes to allow the tissue fragments, now referred to as “explants”, to attach to the plate. Once the explants have attached, enough CEM is added to the plate to cover the explants, and the plates are returned to the incubator.

After a period of 8 or more days, a layer of stromal-like cells begins to arise from adherent explants, covering the surface of the plate surrounding the explant. Over this layer a population of small, round, phase-bright cells is seen. Once the stromal cell layer becomes confluent and there is a large population of bright phase cells, the loosely-adherent cells surrounding the explants are harvested. This is performed by first washing the plate with Ca⁺⁺—Mg⁺⁺-free PBS, then with 0.48 mmol/L EDTA (for 1-2 min) and finally with 0.05% trypsin-EDTA (for 2-3 min). All washes are performed at room temperature under visual control to determine when the loosely adherent cells have become detached. After each step the wash fluid is collected and pooled with that from the other steps. After the final wash, the explants are covered again with CEM and returned to the incubator. Each plate of explants may be harvested in this manner for up to four times at 5-10 day intervals. The pooled wash fluid is then centrifuged at 1000 rpm for 6-8 minutes, forming a cellular pellet. When centrifugation is complete, the supernatant is removed, the pellet is resuspended, and the cells are counted using a hemacytometer. The cells are then plated in poly-d-lysine coated 24-well tissue culture plates at a density ranging from 3-5×10⁴ cells/well (depending on the species) and returned to the incubator. The cells may be grown in either “Cardiosphere Growth Media” (CGM) consisting of 65% Dulbeco's Modified Eagle Media 1:1 with Ham's F-12 supplement and 35% CEM with 2% B27, 25 ng/mL epidermal growth factor, 80 ng/mL basic fibroblast growth factor, 4 ng/mL Cardiotrophin-1 and 1 Unit/mL thrombin, or in CEM alone.

In either media, after a period of 4-28 days, multicellular clusters (“cardiospheres”) will form, detach from the tissue culture surface and begin to grow in suspension. When sufficient in size and number, these free-floating cardiospheres are then harvested by aspiration of their media, and the resulting suspension is transferred to fibronectin-coated tissue culture flasks in CEM (cells remaining adherent to the poly-D-lysine-coated dishes are not expanded further). In the presence of fibronectin, cardiospheres attach and form adherent monolayers of “Cardiosphere-Derived Cells” (CDCs). These cells will grow to confluence and then may be repeatedly passaged and expanded as CDCs, or returned to poly-d-lysine coated plates, where they will again form cardiospheres. Grown as CDCs, millions of cells can be grown within 4-6 weeks of the time cardiac tissue is obtained, whether the origin of the tissue is human, porcine or from rodents. When collagenase is used, the initial increase in cells harvested per mass of explant tissue results in faster production of large numbers of CDCs.

### Example 3

### Exemplary In Vitro Assays for Determination of CDC Properties Following Contact with Various Combinations of Effectors

Human CDCs are treated for 48-72 hours according to the following:
Group I—thymosin β4.
Group II—thymosin β4 and periostin.
Group III—periostin.
Group IV—thymosin β4 and adenosine.
Group V—thymosin β4, periostin and adenosine.
Group VI—CDCs transfected with a vector comprising gene encoding ISL-1
Group VII—adenosine.
Group VIII—adenosine plus CDCs transfected with a vector comprising a gene encoding ISL-1.
Group IX—periostin and adenosine.
Group X—CDCs alone.

### Matrigel Angiogenesis Assay

Angiogenesis of CDCs is assessed by Matrigel in vitro angiogenesis. Briefly, the gel solution is transferred to each well of a pre-cooled tissue culture plate and incubated at 37° C. for at least one hour to allow the gel solution to solidify. CDCs are harvested, resuspended in media and seeded onto the surface of the polymerized Matrigel. Next, CDCs are incubated at 37° C. in the presence or absence of various concentrations of agents described above. Morphological change of the cells is observed at 4, 8 and 12 hours under an inverted light microscope. Patterns of CDCs are recorded and compared with the initial CDC pattern throughout the experiment. The total capillary length and number of branching points are observed and quantified in several random view-fields (3-10) per well. Optionally, cells are stained with commercially available cell stains such as Wright-Giemsa stain crystal violet, or Masson's trichrome to facilitate visualization of cellular networks.

### CDC Migration Assay

In vitro CDC migration is performed using a modified Boyden chamber assay. Briefly, serum-starved CDCs are loaded into the upper compartment of a 96-well microchemotaxis chamber where they are allowed to migrate through the pores of a membrane (e.g., Matrigel coated PET membrane) into the lower compartment. Various concentrations of the agents described above are added to the lower chamber. The membrane between the two compartments is fixed and stained after 4, 8, 12, 18 and 24 hours. The number of cells that have migrated to the lower side of the membrane is determined.

### CDC Survival Assay

In vitro CDC survival is assessed by the WST-1 survival assay. The WST-1 assay is a colorimetric assay based on the cleavage of the tetrazolium salt WST-1 to formazan by cellular mitochondrial dehydrogenases. Cell proliferation results in an increase in the overall activity of the mitochondrial dehydrogenases in the sample, corresponding to an increase in formazan dye metabolism. Briefly, on day 1, WST-1 is added to cells in the various groups described above. Cells are incubated for 3-4 hours under normoxic or hypoxic conditions (1%, 2% or 4% O2). The formazan dye produced by the viable cells is measured at an absorbance of 440 nm using a standard multiwell spectrophotometer each day for up to one week. The extent of cell proliferation is calculated relative to day 1, based on absorbance readings for each sample collected on each day.

### CDC Apoptosis Assay

The apoptosis of CDCs is assessed using known methods, such as by terminal deoxy-nucelotidyl transferase mediated dUTP nick end-labeling (TUNEL) assay for labeling DNA breaks with fluorescent tagged deoxyuridine triphosphate nucleotides (F-dUTP) and total cellular DNA to detect apoptotic cells by flow cytometry or laser scanning cytometry. The enzyme terminal deoxynucleotidyl transferase (TdT) catalyzes a template independent addition of deoxyribonucleoside triphosphates to the 3′-hydroxyl ends of double- or single-stranded DNA. In brief, CDCs treated in the various groups described above are washed with buffer, resuspended, and added to microtiter plate. Fresh 4% paraformaldehyde in PBS is added to the cells, which are then incubated 30 minutes at room temperature on a shaker. Subsequently, the plate is centrifuged for 10 minutes and the supernatant is removed. Cells are resuspended in permeabilization buffer and incubated with TUNEL reaction mixture for an hour at 37° C. until analysis.

### Example 4

### Administration of Effectors and CDCs in Mouse Infarction Model

Male C57B1/6 mice 22-28 g (Jackson Laboratory) undergo anesthesia, analgesia, tracheal intubation, pulmonary ventilation (2 cm H₂0 pressure, 120 min⁻¹, IITC Life Science, Woodland Hills, Calif.), intercostal thoracotomy and ligation of the left anterior descending (LAD) coronary artery (7-0 monofilament suture, Ethicon) to create experimental myocardial infarction. The mice are separated into groups receiving one of the following treatment regimens injected into the coronary artery, or alternatively the myocardium, immediately after ligation:
Group I—thymosin β4 plus CDCs (optionally pre-treated for 48-72 hours with thymosin β4).
Group II—thymosin β4 plus periostin plus CDCs (optionally pre-treated for 48-72 hours with thymosin β4 and/or periostin).
Group III—periostin plus CDCs (optionally pre-treated for 48-72 hours with periostin).
Group IV—thymosin β4 plus adenosine plus CDCs (optionally pre-treated for 48-72 hours with thymosin β4 and/or adenosine).
Group V—thymosin β4 plus periostin plus adenosine plus CDCs (optionally pre-treated for 48-72 hours with thymosin β4, periostin and/or adenosine).
Group VI—CDCs transfected with a vector comprising gene encoding ISL-1
Group VII—adenosine plus CDCs (optionally pre-treated for 48-72 hours with adenosine).
Group VIII—adenosine plus CDCs transfected with a vector comprising a gene encoding ISL-1 (optionally pre-treated for 48-72 hours with adenosine and/or ISL-1).
Group IX—CDCs alone.

A sham surgery control group, undergoes all procedures described except ligation of the LAD. ECG and rectal temperature are monitored intra-operatively. The animals are recovered overnight in a 37° C. environment. The surgeries are performed as part of an institutionally approved protocol. The animals are euthanized at 2, 7 or 14 days. (n=5 for MI and sham groups, at each time point) for harvest of cardiac tissue. Alternatively, the animals are monitored for a period of days following injection, for example, by echocardiography (e.g., to measure left ventricular end systolic dimension (LVESD), left ventricular end diastolic dimension (LVEDD), fractional shortening (FS=100×LVEDD-LVESD/LVEDD) and heart rate) or magnetic resonance imaging (MRI) (e.g., to measure left ventricular volumes at end systole and end diastole (LVESV, LVEDV), left ventricular mass (LVmass), left ventricular ejection fraction (LVEF=LVED-LVESV/LVEDV×100), and left ventricular wall thickening.

The removed cardiac tissue can be subjected to routine histological or immunocytochemical analysis. For example, the cardiac tissue can be fixed and vibratome-sectioned to 1 mm-5 mm thickness, and the resulting sections uniformly processed and paraffin embedded for histology. Some of the sections are stained with hematoxylin-eosin and picrosirius red/fast green to determine, e.g., infarct size. Immunohistochemistry can be performed, e.g., with antibodies directed to various muscle antigens, cardiac antigens or other cell-type antigens.

In certain embodiments, animals in Group II (thymosin plus periostin) will have improved cell engraftment and cardiac function as compared to Groups I (thymosin) III (periostin) and IX (CDCs). In other embodiments, animals in Group IV (thymosin plus adenosine) will have improved cell engraftment and cardiac function as compared to Groups I (thymosin), VII (adenosine) and IX (CDCs). In yet other embodiments, animals in Group V (thymosin plus periostin plus adenosine) will have improved cell engraftment and cardiac function as compared to Groups I (thymosin), III (periostin), VII (adenosine) and IX (CDCs). Finally, in still other embodiments, animals in Group VIII (adenosine plus ISL1) will have improved cell engraftment and cardiac function as compared to Groups VI (ISL1), VII (adenosine) and IX (CDCs).

### Example 5

### Transfection of CDCs and Injection in Mouse Infarction Model

CDCs (10⁶) are transiently cotransfected with pcDNA3 vectors alone or inserted with (i) thymosin β4, (ii) periostin, or (iii) thymosin β4 and periostin via Lipofectamine 2000. CDCs can be further incubated in the presence or absence of adenosine.

Myocardial infarction is created in adult male mice 10 to 20 weeks of age under an approved animal protocol similar to that described in Example 4. Transiently transfected CDCs (10⁵) are injected in a volume of 10 μL of phosphate-buffered saline (PBS) (5 μL at each of 2 sites bordering the infarct), with 10⁵ adenovirally transduced human skin fibroblasts or 10 μL of PBS as controls. Echocardiographs of the mice are taken before the infarction, before the cell injection and 20 days after infarction. The animals are euthanized at 2, 7 or 14 days for harvest of cardiac tissue. Alternatively, the animals are monitored for a period of days following injection, for example, by echocardiography or MRI as described in Example 4.

### Example 6

### Sequential Administration of CDCs and Effectors

To assess engraftment and cell migration, mice are injected with CDCs, either with or without in vitro or ex vivo pretreatment with 100 μg of agents at the time points indicated below (A: adenosine; T: Thymosin β4; P: periostin):

| Time point 1 | Time point 2 | Time point 3 |
|---|---|---|
| (0 min) | (10 min) | (15 min) |
| CDCs, A, T and/or P | — | — |
| CDCs | T and/or P | A |
| CDCs | A | T and/or P |
| CDCs | A, T and/or P | — |
| CDCs | — | A, T and/or P |
| CDCs, A, T and P | — | — |

Control mice receive a non-stem cell, such as fibroblasts, or PBS. After injection, the mice are sacrificed at each of 3 time points (e.g., 0, 8, and 20 days following injection), and the distribution of injected cells is assessed using known methods. Masson's trichrome-stained sections can also be used to quantify regeneration.

### Example 7

### Administration of Effectors and Human CDC in SCID Mouse Infarction Model

Myocardial infarction is created by ligation of the LAD coronary artery in the SCID mice. Human CDCs are prepared and cultured using protocols described in Example 2. Immediately after LAD ligation, one of the following treatment regimes are administered to the mice according to their assigned groups:
Group I—intracardiac injection of 10⁵ human CDCs in 10 μL PBS (optionally pre-treated with thymosin β4 for 48-72 hours).
Group II—intraperitoneal injection of 50 μg thymosin β4 in 300 μL PBS (optionally repeated every 3 days for up to 2 weeks).
Group III—intracardiac injection of 10 μg thymosin β4 in 100 μL PBS (optionally repeated every 3 days for up to 2 weeks).
Group IV—treatment regimes of Group I plus Group II.
Group V—treatment regimes of Group I plus Group III.
Group VI—intracardiac injection of 10 μl PBS ant intraperitoneal injection of 300 μl of PBS at the time of surgery (optionally repeated every 3 days for up to 2 weeks).

### Functional Evaluation

The cardiac functional evaluation of experimental mice is assessed by mouse echocardiography in awake or anesthetized mice with chest hair removed at day 1, weeks 3 and 6 post-MI. Limb leads are attached for electrocardiogram gating, and the animals are imaged in the left lateral decubitus position with a 13-MHz linear probe. Two-dimensional images are recorded in parasternal long- and short-axis projections with guided M-mode recordings at the midventricular level. Left ventricular cavity size and wall thickness are measured at least three beats from each projection and averaged. Left ventricular end systolic dimension, fractional area shortening, LV fractional shortening, relative wall thickness, LV mass, ejection fraction are calculated from the M-mode measurements.

### Human Cell Graft Size

Human CDC graft size is measured by real-time PCR at weeks 3 and 6 following MI procedure using human specific Alu probe. The CDC graft size is assessed by the abundance of Alu, which is quantified using real-time PCR and a standard curve generated by control samples with known number of human CDCs (e.g., 10² to 10⁵) per 12.5 grams of mouse heart tissue.

### Histological Evaluation

The degree of fibrous tissue is assessed at 3 and 6 weeks post MI procedure using Massons trichrome stain. The degree of apoptosis is assessed using a TUNEL assay at 24 hours post-MI procedure. Finally, the degree of inflammatory cell infiltration is assessed using a myeloperoxidase assay at 24 hours post-MI procedure.

### Example 8

### Analysis of Myocardial Regeneration

Horizontal cryosections of 14 μm thickness spaced at 1 mm intervals are analyzed. To determine infarct size, Masson's Trichrome-stained sections are analyzed at I× magnification. The infarct border zone is defined as myocardial tissue within 0.5 mm of the fibrous scar tissue. Fibrosis and cardiomyocyte cross-sectional area are determined after staining with Masson's Trichrome at 10× and 40× magnification, respectively, and quantified using the Metamorph software package. BrdU-positive cardiac fibroblast nuclei are determined at 5 cross-sections per heart at the level of the myocardial infarction. Cardiomyocyte nuclei are counted using the optical dissector method (Howard, CV. & Reed, M. Unbiased Stereology: Three-Dimensional Measurement Jn Microscopy, (BIOS Scientific Publishers, Oxford, 2005)) on troponin T and DAPI-stained sections in 32 - 60 random sample volumes of 84,500 μm per heart. BrdU-positive cardiomyocyte nuclei are quantified on 16-20 sections per heart. Cardiomyocyte apoptosis is determined using the In situ Cell Death Detection Kit (Roche) in combination with staining for troponin I. Capillaries, arterioles, and stem cells are detected with antibodies against von Willebrand factor (vWF), smooth muscle actin (SMA), and c-kit, respectively, and quantified at the level of the myocardial infarction.

### Example 9

### Administration of Effectors and CDCs in Rat Model of Myocardial Infarction

Adult male Sprague-Dawley rats (300 gm, Charles River Laboratories) undergo experimental myocardial infarction as described (del Monte. et al. (2004) Proc Natl Acad Sci USA 101, 5622-7). The survival rate is generally about 67%. Gelfoam® loaded with CDCs (10⁵-10⁹) and simultaneously with 100 μg of the following combinations of agents: (i) adenosine and thymosin β4, (ii) adenosine and periostin, (iii) adenosine, thymosin β4 and periostin or (iv) buffer alone, is applied over the myocardial infarction at the time of surgery. Rats receive 3 intraperitoneal BrdU injections (70 μmol/kg body weight) with a half-life of 2 hr every 48 hr over a period of 7 days. Echocardiography and hemodynamic catheterization are performed as described (Prunier et al. Am J Physiol Heart Circ Physiol (2006)).

### Example 10

### Administration of Effectors and CDCs in Porcine Myocardial Infarction Model

The porcine myocardial infarction is created according to Zuo et al., (2009) Acta Pharmacologica Sinica 30: 70-77. Briefly, pigs are anesthetized with intramuscular diazepam (0.05 mg/kg), atropine (0.05 mg/kg), ketamine (20 mg/kg), intubated. A limited left thoracotomy is performed in a sterile condition through the fifth intercostal space with a small incision in the pericardium. The porcine heart is exposed and suspended in a pericardial sling. A silk suture is set at ⅓ marginal branch of the left anterior descending (LAD) coronary artery and ligated 20 min later. Coronary occlusion is confirmed by the presence of raised ST stages on the electrocardiogram and ventricular arrhythmias within the 1st 20-30 min after occlusion. CDCs and/or effectors are administered to the porcine model according to the procedures described in Example 4.

### Example 11

### Administration of Effectors and CDCs in HumAn Subjects

Patients with chronic or acute heart failure are given the following clinical procedures upon experiencing symptoms of myocardial infarction.

Catheterization is performed by (A) intracoronary doppler (optional) followed by (B) coronary angiography and cell/effector administration. Doppler measurements and coronary angiography are repeated in case that a premature coronary angiography has to be performed for clinical reasons (e.g. restenosis).

### Intracoronary Doppler

### Adenosine Administration

Adenosine (Adenoscan®) intravenously to the patient at a concentration of 140 μg/kg body weight/min at an infusion rate>100 ml/h according to the infusion scheme presented in Table 2:

TABLE 2

| Body Wt. (kg) | ml/min | ml/h |
|---|---|---|
| 45-49 | 2.1 | 126 |
| 50-54 | 2.3 | 138 |
| 55-59 | 2.5 | 150 |
| 60-64 | 2.8 | 168 |
| 65-69 | 3.0 | 180 |
| 70-74 | 3.3 | 198 |
| 75-79 | 3.5 | 210 |
| 80-84 | 3.8 | 228 |
| 85-89 | 4.0 | 240 |
| 90-94 | 4.2 | 252 |
| 95-99 | 4.4 | 264 |
| 100-104 | 4.7 | 282 |
| 105-109 | 4.9 | 294 |
| 110-114 | 5.1 | 306 |
| 115-119 | 5.4 | 324 |

Optionally, thymosin β4 and/or periostin is administered to the patient intravenously or intramyocardially at the discretion of the physician.

### Measurement of Flow Reserve in Infarct Artery

Flow reserve in the infarct artery is measured according to the following procedure. First, the vessel is pretreated with Nitroglycerin 0.2 mg i.c. Flowire is positioned at the site of the stent (target lesion of index infarction), in which the position is documented by coronary angiography. Adenosine infusion begins following documentation of time, heart rate, blood pressure, and APV and continues for further 45 seconds after maximal increase of flow (steady state). Bradycardia is attended to during the time of infusion.

### Measurement of Flow Reserve in Reference Vessel

Flow reserve in reference vessel is measured by the following procedure. First, vessel is treated with Nitroglycerin 0.2 mg i.c., if not already performed in this vessel. Flowire is positioned at the site in a non-diseased portion of the vessel. In this procedure, an ideal reference vessel is a vessel that has not been treated by PCI within the last 6 months, is not significantly diseased and has no previous myocardial infarction in the reference vessel. For this procedure, all three major vessels (RCA, LCX, LAD) or major branches are suitable as a reference vessel. The coronary flow velocity is attended to until it is back to baseline. This procedure is repeated as described above for infarct artery. Angiographic projections are documented for follow-up measurements.

### Preparation and Administration of CDCs

Percutaneous right ventricular endomyocardial biopsy specimens are obtained from patients during previous hospital visits after informed consent using an institutional review board-approved protocol. CDCs are prepared from the specimen and cultured according to protocols described in Example 2. Autologous CDCs are adminstered to the patient following one of the treatment regimes:
Group I—CDCs (optionally pre-treated for 48 hours with thymosin β4) plus thymosin β4.
Group II—CDCs (optionally pre-treated for 48 hours with thymosin β4 and/or periostin) plus thymosin β4 plus periostin.
Group III—CDCs (optionally pre-treated for 48 hours with periostin) plus periostin.
Group IV—CDCs (optionally pre-treated for 48 hours with thymosin β4 and/or adenosine) plus thymosin β4 plus adenosine.
Group V—CDCs (optionally pre-treated for 48 hours with thymosin β4, periostin and/or adenosine) plus thymosin β4 plus periostin plus adenosine.
Group VI—CDCs transfected with a vector comprising gene encoding ISL-1
Group VII—CDCs (optionally pre-treated for 48 hours with adenosine) plus adenosine.
Group VIII—CDCs transfected with a vector comprising a gene encoding ISL-1 (optionally pre-treated for 48 hours with adenosine and/or ISL-1) plus adenosine.
Group IX—CDCs alone

### Premedication

Prior to application of the above treatments, ReoPro® (Abciximab, Bolus only) is given to the patient according to prescription dosage of 0.25 mg/kg body weight over 1 min, with optional subsequent continuous infusion of abciximab at the discretion of the investigator.

Glycoprotein-receptor blocker therapy is recommended at the time of treatment of the acute myocardial infarction by the protocol. However, indication and type of glycoprotein receptor blocker (tirofiban, eptifibatide or abciximab) is left at the discretion of the physician in charge. Nevertheless, in line with current evidence, use of abciximab (ReoPro) is encouraged also at the index PCI. In this case, abciximab will be given as an re-administration during cell therapy. The platelet count is controlled 6 and 24 hours after study therapy as well as prior to hospital discharge for any potential thrombocytopenia. In addition, approximately 50-70 units/kg of heparin are given (target ACT 250-300s) prior to cell/placebo medium therapy.

### Balloon Placement

Balloon placement is performed using a 6 F guiding catheter. For cell infusion, a conventional over-the-wire balloon (Opensail®, Guidant) is used; cell- or placebo-solution are infused through the central guide wire lumen. The balloon is oversized by 0.5 mm compared to the size of the implanted stent to achieve an occlusion of the vessel during low pressure balloon insufflation. Long balloons with a length of 10 mm are used in the procedure. However, if the balloon size is larger than 4 mm, only a 20 mm long balloon is available. Next, a conventional guide wire is inserted in the Opensail® balloon catheter (no long exchange wire is necessary) to advance the balloon to the guide wire tip. The guide wire is then introduced to the infarct vessel. Subsequently, Opensail® balloon catheter is advanced to the previous infarct lesion; the balloon within the stent is positioned.

### Set Up of Infusion

The infusion is set up by retracting the guide wire and connecting a 3-way tap to the central lumen. It is important to remove air from the system before injecting the cells. The central lumen is then flushed with albumin, which lubricates the wall of the balloon catheter and avoids attachment of cells to the wall of the balloon catheter. The syringe containing CDCs (and effectors) according to the treatment regimes or placebo solution is connected with the cell suspension to the 3-way tap.

### Balloon Insufflation and Cell Injection

Balloon insufflation is performed according to the following procedure. Prior to and after this balloon inflation, the patient is given 100 μg adenosine i.v. in repeated boluses up to 1 mg. The vessel is occluded with a low pressure balloon insufflation. It is essential to choose a slightly oversized balloon to prevent the balloon pressure from exceeding 2-4 bars. A few ml of contrast agent is injected with care not to damage the occluded artery, with documentation that the vessel is actually occluded before giving the cells. A complete occlusion by coronary angiography is recorded. If the vessel is not occluded, the balloon should be expanded and holds the 2-4 bar pressure. If the vessel is still not occluded despite adequate balloon expansion, a larger balloon is used with care not to exert extensive pressure (>4 bar) on vessel wall. Injection of the progenitor cells is only allowed if complete occlusion has been successfully documented by cine angiography. Balloon occlusion is intended to avoid wash out of the cells and to give the cells time to attach in the target area. It is intended that the infarct artery is occluded for 3 minutes. Occlusion is checked immediately by angiography; long delays between balloon occlusion and actual start of infusion of the study therapy should be avoided to maximize time for cells to home in the infarct area. Thereafter, one-third of the solution in the syringes (3.3 ml) is injected within 10 seconds. The balloon is deflated after 3 minutes. In case of severs angina pectoris, the balloon might be deflated earlier. However, patients after myocardial infarction can generally tolerate a three-minute occlusion without or with only minor chest pain. The actual time of sufficient balloon inflation after infusion of the cells is documented. Three minutes after deflation of the balloon, this procedure is repeated for two additional times. Finally, the balloon catheter is removed; the integrity of the infarct artery by coronary angiography is recorded. An overview angiography (RAO 30°; LAO)60° is performed additionally without zoom for documentation of the absence of microembolization. The schedule for cell and effector infusion is summarized in Table 3.

TABLE 3

| | |
|---|---|
| Balloon inflation | |
| Angiography to document occlusion | immediately after sufficient |
| | inflation |
| Infusion of cells and effectors | immediately after angiography |
| | (about 10 sec.) |
| Deflation of balloon | after 3 minutes |
| Pause | 3 minutes |
| Second balloon inflation | time schedule as above |
| Pause | 3 minutes |
| Third balloon inflation | time schedule as above |

The clinical procedures presented in this example are given to the patients repeatedly over a course of one year at the discretion of the physician. Echocardiogram, cardiac MRI, a 24-hour Holter monitor and laboratories (including, e.g., complete blood count (CBC), blood urea nitrogen (BUN), creatinine, troponin, lactate dehydrogenase (LDH), c-reactive protein (CRP), and norepinephrine) are performed periodically to assess adverse outcome.

The embodiments of the present invention described above are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims. Furthermore, as used in this specification and claims, the singular forms “a,” “an” and “the” include plural forms unless the content clearly dictates otherwise. Thus, for example, reference to “a positive effector” includes a mixture of two or more such effectors, and the like. Additionally, ordinarily skilled artisans will recognize that operational sequence must be set forth in some specific order for the purpose of explanation and claiming, but the present invention contemplates various changes beyond such specific order.

The contents of all references described herein are hereby incorporated by reference. Other embodiments are within the following claims.

1.-101. (canceled)

102. A method for treating injured cardiac tissue in a subject, comprising:
identifying a subject having injured cardiac tissue;
providing two or more of a positive effector, a negative effector, and an ancillary effector,
wherein said positive effector comprises thymosin beta-4;
contacting said injured cardiac tissue with said thymosin beta-4;
wherein said injured cardiac tissue has a deficiency in one or more of cardiac output, cardiac tissue viability, cardiac blood flow,
wherein said contacting of said thymosin beta-4 with said injured cardiac tissue improves one or more of said cardiac tissue deficiencies, thereby treating said injured cardiac tissue.

103. The method of claim 102, wherein said negative effector is provided and is selected from the group consisting of one or more of the following: adenosine, an adenosine agonist, an adenosine receptor agonist, a phosphoinositide 3-kinase inhibitor, a caspase inhibitor, cyclosporine, an opiod receptor antagonist, pinacidil, a nitric oxide donor, poly(ADP-ribose) inhibitors, sodium-hydrogen exchange inhibitors, and thymosin beta-4.

104. The method of claim 103, wherein said negative effector is characterized by the ability to inhibit or reduce one or more of apoptotic cell death or inflammation.

105. The method of claim 102, wherein said ancillary effector is provided and is selected from the group consisting of one or more of the following: p38 MAP kinase inhibitors, phosphodiesterase inhibitors, stem cell factor, and transforming growth factor beta.

106. The method of claim 105, wherein said ancillary effector promotes one or more of angiogenesis, revascularization, cell-to-cell contact, or cell-to-cell communication

107. The method of claim 105, wherein said ancillary effector is further characterized by the ability to facilitate the effects of positive and/or negative effectors.

108. The method of claim 102, wherein said contacting of said thymosin beta-4 with said injured cardiac tissue activates, enhances, or promotes one or more of proliferation, migration, differentiation, or cell cycle re-entry in the cells of the injured cardiac tissue.

109. The method of claim 102, wherein at least one of said negative effector and said ancillary effector are provided and are different from said positive effector.

110. A method for treating injured cardiac tissue in a subject, comprising:
identifying a subject having injured cardiac tissue;
providing one or more of a positive effector, a negative effector, and an ancillary effector,
wherein the positive effector comprises thymosin beta-4;
providing cardiosphere derived cells (CDCs) harvested from non-embryonic cardiac tissue;
contacting said injured cardiac tissue or said CDCs with said thymosin beta-4 and optionally with one or more of the negative and ancillary effectors; and
contacting said CDCs with said injured cardiac tissue,
wherein said injured cardiac tissue has a deficiency in one or more of cardiac output, cardiac tissue viability, cardiac blood flow; and
wherein said contacting of said CDCs with said injured cardiac tissue improves one or more of said cardiac tissue deficiencies, thereby treating said injured cardiac tissue.

111. The method of claim 110, wherein said injured cardiac tissue is contacted with said thymosin beta-4, resulting in activation, enhancement, or promotion of one or more of proliferation, migration, differentiation, or cell cycle re-entry in the cells of the injured cardiac tissue.

112. The method of claim 110, wherein said CDCs are contacted with said thymosin beta-4, resulting in activation, enhancement, or promotion of one or more of cell proliferation, cell engraftment, cell migration, cell differentiation, or cell cycle re-entry in said CDCs.

113. The method of claim 110, wherein said negative effector is provided and is characterized by the ability to inhibit or reduce one or more of apoptotic cell death or inflammation.

114. The method of claim 110, wherein said ancillary effector is provided and promotes one or more of angiogenesis, revascularization, cell-to-cell contact, or cell-to-cell communication.

115. The method of claim 110, wherein said injured cardiac tissue or said CDCs are individually contacted with said thymosin beta-4 and optionally said negative and/or said ancillary effector prior to being contacted with one another.

116. The method of claim 110, wherein said injured cardiac tissue is sequentially contacted with said CDCs followed by one or more of said thymosin beta-4, said negative effector, and said ancillary effector.

117. The method of claim 110, wherein said injured cardiac tissue is sequentially contacted one or more of said thymosin beta-4, said negative effector, and said ancillary effector followed by said CDCs.

118. The method of claim 110, wherein the source of said CDCs is autologous relative to the subject having injured cardiac tissue.

119. The method of claim 110, wherein the source of said CDCs is allogeneic relative to the subject having injured cardiac tissue.

120. A composition for treating injured cardiac tissue in a subject, comprising:
non-embryonic cardiac stem cells,
wherein said stem cells are cardiosphere-derived cells; and
thymosin beta-4,
wherein said thymosin beta-4 is characterized by the ability to activate, enhance, or promote one or more of cell proliferation, cell engraftment, cell migration, cell differentiation, or cell cycle re-entry; and
wherein said composition is suitable for treating injured cardiac tissue that has a deficiency in one or more of cardiac output, cardiac tissue viability, cardiac blood flow.

121. The composition of claim 120, further comprising a negative effector and an ancillary effector,
wherein said negative effector is characterized by the ability to inhibit or reduce one or more of apoptotic cell death or inflammation,
wherein said ancillary effector promotes one or more of angiogenesis, revascularization, cell-to-cell contact, or cell-to-cell communication, and
wherein said negative effector and said ancillary effector are not thymosin beta-4 and are different from one another.
